SEQUENCE LISTING (13 sequences)

SEQ ID NO: 1 - RNA, 25 nt, Homo sapiens
gacaguggaaguuuuuuuuuccucg

SEQ ID NO: 2 - RNA, 19 nt, Homo sapiens
auuagugucaucuugccuc

SEQ ID NO: 3 - RNA, 25 nt, Homo sapiens
aaugcccuacaucuuauuuuccuca

SEQ ID NO: 4 - RNA, 24 nt, Homo sapiens
gguucaagcgauucucgugccucg

SEQ ID NO: 5 - RNA, 24 nt, Homo sapiens
ggcugguccgaacuccugaccuca

SEQ ID NO: 6 - RNA, 21 nt, Homo sapiens
gauucacccaccuuggccuca

SEQ ID NO: 7 - RNA, 28 nt, Homo sapiens
ggguguuaagacuugacacaguaccucg

SEQ ID NO: 8 - RNA, 28 nt, Homo sapiens
agugcuuaugaggggauauuuaggccuc

SEQ ID NO: 9 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 10 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 11 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 12 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 13 - RNA, 21 nt, Homo sapiens
gaugcacccaccuuggccuca

(19) United States
(12) Patent Application Publication
(10) Pub. No.: US 2020/0325234 A1
(43) Pub. Date: Oct. 15, 2020

(54) IMMUNE-BASED TREATMENT OF KRAS-VARIANT CANCER PATIENTS

(71) Applicant: MIRA DX, INC., Los Angeles, CA (US)

(72) Inventor: Joanne Weidhaas, Los Angeles, CA (US)

(73) Assignee: MIRADX, Los Angeles, CA (US)

(21) Appl. No.: 16/096,316

(22) PCT Filed: Apr. 27, 2017
(86) PCT No.: PCT/US2017/029938

§ 371 Date: Oct. 25, 2018

Related U.S. Application Data
(60) Provisional application No. 62/328,548, filed on Apr. 27, 2016.

(51) Int. Cl. C07K 16/28; C12Q 1/6886

(52) U.S. Cl. CPC C07K 16/2863; C12Q 1/6886; C12Q 2600/106; A61K 45/06; C07K 16/2827; C07K 16/2818; C12Q 2600/156

(57) ABSTRACT

The present invention is directed to methods of administering an immunotherapy, in combination with other conventional cancer treatments, to a cancer patient wherein said administration is dependent on the presence of a KRAS-variant. The invention further provides diagnostic methods for determining the increased likelihood that a cancer patient will respond to an immunotherapy based on the presence of the KRAS-variant.

### CROSS-REFERENCE TO RELATED APPLICATIONS

This application claims priority to and the benefit of U.S. Provisional Patent Application No. 62/328,548 filed Apr. 27, 2016, the entirety of which is incorporated by reference herein.

### FIELD OF THE INVENTION

The present invention is directed to methods of administering an immune modulating agent to a patient, in need thereof, wherein the choice of the immune modulating agent is dependent on the presence of a KRAS-variant. In one aspect of the invention, a method is provided of administering to a cancer patient an immune modulating agent designed to enhance the immune system, in combination with other conventional cancer treatments. In a preferred embodiment, an agent is administered to a cancer patient in combination with radiation treatment. The invention further provides diagnostic methods for determining the increased likelihood that a cancer patient, or autoimmune patient, will respond to a specific immunotherapy based on the presence of the KRAS-variant.

### BACKGROUND OF THE INVENTION

The KRAS-variant is a biologically functional, microRNA binding site variant in the KRAS oncogene, which predicts increased cancer risk. MicroRNA (miRNA) binding site variants in the 3′ untranslated region (3′UTR) of important growth and survival genes are a recently discovered novel class of germ-line mutations, which are powerful biomarkers of cancer risk and treatment response (Cipollini et al. (2014) PHARMGENOMICS PERS MED 7: 173-191).

One of the first mutations discovered in this class is the KRAS-variant, a let-7 binding site mutation in the 3′UTR of the KRAS oncogene (Chin et al. (2008) CANCER RES 68:8535-40). This mutation predicts an increased risk of several cancers, including non-small cell lung cancer (Id.), triple negative breast cancer (TNBC) in premenopausal women (Paranjape et al. (2011) THE LANCET ONCOLOGY 12(4):377-386) and ovarian cancer (Ratner et al. (2010) CANCER RESEARCH 15:6509-15; Ratner et al. (2012) ONCOGENE 31(42):4559-66; Pilarski et al. (2012) PLOS ONE 7(5):e37891). The KRAS-variant has also been shown to predict unique tumor biology, with tumors in KRAS-variant patients exhibiting a KRAS-addicted signature as well as an estrogen-negative, basal-like gene expression pattern (Ratner, 2012, supra; Paranjape, supra). Women with the KRAS-variant have also been found to be at a significantly increased risk of developing multiple primary cancers, including breast and ovarian cancer, as well as a third independent cancer in the same individual (Pilarski, supra).

Substantial evidence that the KRAS-variant acts as a cancer biomarker of response to therapy also exists. This includes cisplatin resistance in KRAS-variant patients with ovarian or head and neck cancer (Ratner, 2012, supra; Chung et al. (2014) ANN ONCOL, July 31. [Epub ahead of printing]), cetuximab sensitivity in KRAS-variant patients with colon cancer (Saridaki et al. (2014) CLIN CANCER RES 20(17):4499-510) or head and neck cancer (Chung, supra), and erlotonib resistance but sorafenib sensitivity in KRAS-variant patients with non-small cell lung cancer (NSCLC) (Weidhaas et al. (2014) J CLIN ONCOL 32(52):suppl; abstr 8135). Cell line data further supports the unique response of the KRAS-variant to chemotherapy exposures (Saridaki, supra).

Immunotherapeutic approaches to treatment of cancer have been practiced previously. For example, in cancer treatments, attempts have been made to elicit an immune response to the cancer itself. Such treatments typically involve, for example, administration of antibodies, including monoclonal antibodies and fragments thereof, that specifically recognize and target destruction of cancer cells, administration of immunocytokines or checkpoint inhibitors designed to stimulate the immune system, and administration of autologous or allogeneic immune cells, which in some instances have been genetically engineered to enhance their immune function, and which are expected to elicit a successful immune response to cancer cells. Accordingly, there is a need in the art for methods to prevent and treat cancer in subjects with the KRAS-variant. In addition, there is a need in the art for methods of predicting those cancer subjects likely to respond to a specific immunotherapy so that the correct treatment is appropriately administered. There is also a need to identify patients who will or will not experience toxicity from such treatments in order to appropriately manage or direct such treatments.

### SUMMARY OF THE INVENTION

The present invention relates to the discovery that subjects with the KRAS-variant have altered immune systems. Specifically, as described herein, KRAS-variant subjects have been shown to possess weakened immune systems, relied on for the successful treatment of cancer. As described herein such immune systems can be enhanced, or stimulated, by administration of an appropriate immune modulating agent.

Accordingly, in one aspect, the present invention is directed to methods of administering a specific immune modulating agent to a cancer patient in the presence of a KRAS-variant. In a specific embodiment, the immune modulating agent is administered in combination with other conventional cancer treatments. In such an instance, the KRAS-variant subject is treated, will be treated, or has been pre-treated, with one or more conventional cancer treatments comprising, for example, chemotherapy, radiotherapy, or surgery. More specifically, the invention provides a method for treating a KRAS-variant cancer subject, having a single nucleotide polymorphism (SNP) at position 4 of the let-7 complementary site 6 of KRAS comprising administration of an immune modulating agent wherein said subject is treated, will be treated or has been pre-treated with one or more therapies comprising chemotherapy, radiotherapy, or surgery.

In a specific embodiment of the invention, radiation treatment may function as a sensitizer to further treatment with an immune modulator, and the administration of an immune modulating agent such as cetuximab can lead to a further enhancement of the immune response directed against cancer cells. Accordingly, in a specific embodiment of the invention radiation therapy is co-administered with, for example, anti-cancer antibodies such as cetuximab or panitumimab to a KRAS-variant cancer subject. Other options may include T cell therapy, or other immune enhancing therapy.

The method further comprises detecting a single nucleotide polymorphism (SNP) at position 4 of the let-7 complementary site 6 of KRAS in a patient sample wherein the presence of said SNP indicates an increased beneficial effect associated with administration of a specific immune modulating agent for said subject. Further, the presence of the KRAS-variant indicates an increased sensitivity to radiotherapy in normal tissue, but a lack of a systemic immune response, resulting in the development of metastatic disease, indicating the usefulness of co-administration of immune enhancement with radiation therapy. In an embodiment of the invention, the cancer includes, but is not limited to, any cancer treated with radiation, including for example, breast cancer, ovarian cancer, non-small and small cell lung cancer, colorectal cancer, pancreatic cancer, brain cancer, gastric cancer, uterine cancer, testicular cancer, sarcoma, prostate cancer, lymphomas and head and neck cancer. The invention also provides methods for determining whether a cancer subject will likely respond to administration of a specific immune modulating agent based on the presence of the KRAS-variant. Specifically, the present invention is directed to methods of selecting a specific immune modulating agent to a patient, in need, thereof, wherein the choice of the immune modulating agent to be administered is dependent on the presence of a KRAS-variant. In the presence of the KRAS-variant, immune modulating agents that function to initially stimulate a weakened immune system are preferred over agents which rely on a fully functional immune system for their benefit. Immune modulating agents to be administered to KRAS-variant patients include, for example, antibodies, cytokines, adoptive cell transfer, while those agents such as checkpoint inhibitors are less preferred. More specifically, the invention provides a method of predicting an increased beneficial effect of such administration for a KRAS-variant cancer subject, comprising detecting a single nucleotide polymorphism (SNP) at position 4 of the let-7 complementary site 6 of KRAS in a patient sample wherein the presence of said SNP indicates an increased beneficial effect resulting from immunotherapy. In addition, the presence of the KRAS-variant may indicate an increased beneficial effect associated with co-administration of an immune modulator and radiation therapy. The present invention has significant clinical value, as the method provides a means for identifying whether a cancer subject is likely to respond to administration of an immune modulating agent. A patient identified as having a KRAS-variant is identified as likely to respond to immunotherapy and a patient who does not have the KRAS-variant is identified as unlikely to respond to administration of an immune modulating agent. Thus, if a patient is positive for the KRAS-variant, the doctor is provided with a means for choosing an optimal treatment while avoiding an ineffective treatment.

In addition, the present invention provides a means for identification of a suitable target patient, or target subpopulation of patients, for clinical trial design. In a specific embodiment of the invention, the presence of the KRAS-variant indicates that a certain target population should receive one type of immunotherapy versus another. Accordingly, subjects having the KRAS-variant may be chosen for clinical trials wherein said treatment involves administration of a drug, or treatment, designed to stimulate, or enhance, the immune system while those agents which rely on a fully functional immune system for their benefit are less preferred. Alternatively, subjects having the KRAS-variant may be chosen for clinical trials wherein the efficacy of a test drug is enhanced by co-administration of an immune modulating agent. Such a targeted selection of test subjects may serve to streamline the drug approval process by reducing the size and numbers of trials thereby facilitating quick regulatory approval and advancement of the drug to market.

In instances where it is found that the presence of the KRAS-variant is associated with increased efficacy of a tested drug or treatment, the present invention further provides methods for testing of a patient for the presence of the KRAS-variant prior to prescribing of the tested/approved drug by a physician. In such instances, the drug label may contain instructions that the patient should be tested for presence of the KRAS-variant prior to administration of the drug or treatment. Accordingly, the present invention is also directed to a combination drug label wherein said label refers to the use of a drug which as a condition of use must be used in combination with a diagnostic test wherein said diagnostic test is designed to detect the presence of a KRAS-variant in said subject. More specifically, the invention provides diagnostic methods for testing of a patient prior to prescribing of a drug, and to combination drug labels, wherein the diagnostic test comprises detecting a single nucleotide polymorphism (SNP) at position 4 of the let-7 complementary site 6 of KRAS in a patient sample wherein the presence of said SNP indicates an increased beneficial effect resulting from administration of an immune modulating agent.

In another embodiment, the invention provides a reduced-toxicity method of treating cancer where an immune-modulating cancer therapy is administered to a cancer subject who has been determined to have a single nucleotide polymorphism (SNP) at position 4 of the let-7 complementary site 6 of KRAS. According to this invention, the immune-modulating cancer therapy is radiation, whereas in another embodiment, the immune-modulating cancer therapy is a checkpoint inhibitor, for example, an anti-PDL 1 or anti-PD1 antibody therapy.

The invention also provides a method of predicting the toxicity of an immune-modulating cancer therapy in a subject. In this method, one detects the presence or absence of a single nucleotide polymorphism (SNP) at position 4 of the let-7 complementary site 6 of KRAS in a nucleic acid from the subject. If the polymorphism is detected in the patient, it is indicative of a reduced likelihood of toxicity of the immune-modulating cancer therapy in the subject. According to one embodiment of this invention, the immune-modulating cancer therapy may be radiation, whereas in another embodiment, the immune-modulating cancer therapy may be a checkpoint inhibitor, for example, an anti-PDL 1 or anti-PD1 antibody therapy.

### BRIEF DESCRIPTION OF THE DRAWINGS

This application file contains at least one drawing executed in color. Copies of this patent or patent application publication with color drawing(s) will be provided by the Office upon request and payment of the necessary fee.

FIG. 1A-D. Progression-free survival (PFS), local-regional failure (LRF), distant metastasis (DM) and overall-survival (OS) for HNSCC patients by KRAS-variant status and assigned treatment. In total, 179 of 413 patients experienced a PFS failure (FIG. 1A): 19 of 38 in the non-cetuximab-treated KRAS-variant group, 13 of 32 in the cetuximab-treated KRAS-variant group, 74 of 169 in the non-cetuximab-treated non-variant group, and 73 of 174 in the cetuximab-treated non-variant group. In total, 97 of 413 patients experienced local-regional failure (FIG. 1B): 8 of 38 non-cetuximab-treated KRAS-variant group, 6 of 32 in the cetuximab-treated KRAS-variant group, 39 of 169 in the non-variant non-cetuximab-treated group, and 44 of 174 in the non-variant cetuximab-treated group. In total, 51 of 413 patients have experienced distant metastasis (FIG. 1C): 8 of 38 in the non-cetuximab-treated KRAS-variant group, 3 of 32 in the cetuximab-treated KRAS-variant group, 21 of 169 in the non-variant non-cetuximab-treated, and 19 of 174 in the non-variant cetuximab-treated group. In total, 134 of 413 patients died (FIG. 1D) within the relevant time frame: 14 of 38 in the non-cetuximab-treated KRAS-variant group, 8 of 32 in the cetuximab-treated KRAS-variant group, 58 of 169 in the non-variant non-cetuximab-treated group, and 54 of 174 in the non-variant cetuximab-treated group.

FIG. 1E. Local Failure for KRAS-variant patients by p16 status and cetuximab treatment. HPV positive (solid lines) versus negative (dashed lines). Black lines represent cetuximab treatment. Red lines (marked with solid circles) represent no cetuximab. Higher LRF was observed for HPV positive patients (red solid versus dashed), with no benefit of cetuximab. Better local control for p16 negative patients was observed with a benefit of cetuximab with no LRF (dotted black).

FIG. 1F. Distant metastases for KRAS-variant patients by p16 status and cetuximab treatment. HPV positive (solid lines) versus negative (dashed lines). Black lines represent cetuximab treatment. Red lines (marked with solid circles) represent no cetuximab. Higher distant metastases were observed without cetuximab for both HPV positive (solid red) and HPV negative (dotted red). The impact of cetuximab is affected by time for p16-negative.

FIG. 2A-B: Progrssion-Free Survival by KRAS Genotype and p16 Status for Patients treated without or with Cetuximab Treatment. Solid lines are KRAS-variant patients, dotted lines non-variant, black lines represent p16 positive, red lines (marked with solid circles) represent p16 negative. (FIG. 2A) PFS without cetuximab. KRAS-variant/p16 positive patients (black solid line) do poorly compared to non-variant p16 positive patients (block dotted line), and KRAS-variant p16 negative patients (red solid line) have improved outcome compared to non-variant/p16 negative patients (red dotted line). (FIG. 2B) PFS with 8 weeks of cetuximab. KRAS-variant/p16 positive patients (black solid line) have similar PFS to non-variant p16 positive patients (black dotted line) that lasts through the five years of follow up. KRAS-variant/p16 negative patients initially have improved PFS that falls off after year three.

FIG. 3A-B. Overall survival by KRAS Genotype and p16 Status for Patients treated without or with Cetuximab Treatment. Solid lines are KRAS-variant patients, dotted lines non-variant, black is p16 positive, red (marked with solid circles) is p16 negative. (FIG. 3A) OS without cetuximab. KRAS-variant/p16 positive patients (black solid line) do poorly compared to non-variant/p16 positive patients (block dotted line), and KRAS-variant/p16 negative patients (red solid line) have improved outcome compared to non-variant/p16 negative patients (red dotted line). (FIG. 3B) OS with 8 weeks of cetuximab. KRAS-variant/p16 positive patients (black solid line) have improved OS that lasts through the five years of follow up. KRAS-variant/p16 negative patients have initial improved OS that falls off after year three.

FIG. 4A-B. Immune profiling of KRAS-variant versus non-variant HPV positive HNSCC patients. (FIG. 4A) Immune profiling evaluated Lymphoid and Myeloid subsets, and CD4 and CD8 subsets are also shown. Significant differences are found in KRAS-variant patients (red-labeled with No. 2), with higher CD4+ cells, primarily effector cells, borderline lower PD 1+CD8 cells, an altered CD4/CD8 ratio, and lower NK cells. Myeloid subsets are also significantly altered. (FIG. 4B) Graphic depiction of differences between KRAS-variant (solid line) and non-variant (dotted line) patients.

FIG. 5. Double strand break repair and the KRAS-variant. In normal tissue cell lines (MCF 10A, P=parent, M=KRAS-variant the variant is associated with higher baseline DS damage and slower repair after irradiation. In tumor cell lines (H1299, P=parent, M=KRAS-variant, the variant is associated with less baseline DS damage and faster repair after irradiation. These findings indicate that KRAS-variant patients' normal tissues will be sensitive to radiation, while their tumor tissues may be resistant.

FIG. 6. Radiosensitivity of KRAS-variant cell lines. Clonogenic assays were performed to evaluate radiosensitivity in two pairs if isogenic cell lines, MCF 10A and H1299. Variant=KRAS-variant, and non-variant=parental line. Error bars represent Standard Deviation between replicates shown as percentage. The normal tissue KRAS-variant line is more sensitive.

### DETAILED DESCRIPTION

### Introduction

The KRAS-variant, a SNP in the 3′ untranslated region (UTR) of KRAS, referred to herein as the “LCS6 SNP,” or the “KRAS-variant,” is a germ-line, dynamically regulated microRNA binding site mutation in the KRAS oncogene, which predicts increased likelihood of a cancer patient responding to administration of an immune modulating agent. The invention is based upon the unexpected discovery that subjects having the KRAS-variant have altered immune systems. Specifically, as described herein, KRAS-variant subjects are shown to possess a weakened immune system, normally relied upon for destruction of cancer cells, that can be enhanced, or stimulated, by administration of an immune modulating agent for treatment of cancer.

Accordingly, the invention provides methods for determining whether a cancer subject is likely to respond beneficially to administration of a specific immune modulator based on the presence of the KRAS-variant. Specifically, the present invention is directed to methods of selecting a specific immune modulating agent to be administered to a patient in need thereof, wherein the choice of the immune modulating agent to be administered is dependent on the presence of a KRAS-variant. In the presence of the KRAS-variant, immune modulating agents that function to initially stimulate a weakened immune system are preferred over agents which rely on a fully functional immune system for their benefit. Immune modulating agents to be administered to KRAS patients include, for example antibodies, cytokines, adoptive cell transfer, while those agents such as checkpoint inhibitors are less preferred. More specifically, the invention provides a method of predicting an increased beneficial effect of an immunotherapy for a KRAS-variant cancer subject, comprising detecting a single nucleotide polymorphism (SNP) at position 4 of the let-7 complementary site 6 of KRAS in a patient sample wherein the presence of said SNP indicates an increased beneficial effect resulting from immunotherapy. The present invention has significant clinical value, as the method provides a means for identifying whether a cancer subject is more likely to respond to administration of one specific immune modulating agent than another. A patient identified as having a KRAS-variant is identified as likely to respond to immunotherapy and a patient who does not have the KRAS-variant is identified as unlikely to respond to immunotherapy (in the absence of, for example, an adjunctive therapeutic regimen to assist in the initiation of an immune response to the tumor). For certain immune modulating agents, such as checkpoint inhibitors, a cancer patient identified as having a KRAS-variant is less likely to respond to the therapy (in the absence of, for example, an adjunctive therapeutic regimen to assist in the initiation of an immune response to the tumor) than a patient who does not have the KRAS-variant. Thus, if a patient is tested for the KRAS-variant, the doctor is provided with a means for choosing an optimal treatment while avoiding an ineffective treatment.

In addition, the present invention provides methods for identification of a suitable target patient, or target subpopulation of patients, for clinical trial design. Accordingly, subjects having the KRAS-variant may be chosen for clinical trials wherein said treatment involves administration of a drug, or treatment, designed to stimulate the immune system. Alternatively, subjects having the KRAS-variant may be chosen for clinical trials wherein the efficacy of a test drug may be enhanced by co-administration of an immunotherapy. Such a targeted selection of test subjects may serve to streamline the approval process by reducing the size and numbers of trials thereby facilitating quick regulatory approval and advancement of the drug to market.

In instances where it is found that the presence of the KRAS-variant is associated with increased efficacy of a tested drug, the present invention further provides methods for testing of a patient for the presence of the KRAS-variant prior to prescribing of the tested/approved drug by a physician. In such instances the drug label may contain instructions that the patient should be tested for presence of the KRAS-variant prior to administration of the drug. Thus, the present invention relates to a product drug label wherein said label refers to the use of a drug, or treatment method, which as a condition of use must be used in combination with a diagnostic test wherein said diagnostic test is designed to detect the presence of a KRAS-variant. In such an instance, the presence of the KRAS-variant indicates usage of said drug or treatment.

There are three human RAS genes comprising HRAS, KRAS, and NRAS. Each gene comprises multiple miRNA complementary sites in the 3′UTR of their mRNA transcripts. Specifically, each human RAS gene comprises multiple let-7 complementary sites (LCSs). The let-7 family-of-microRNAs (miRNAs) are global genetic regulators important in controlling cancer oncogene expression by binding to the 3′UTRs (untranslated regions) of their target messenger RNAs (mRNAs).

Specifically, the term “let-7 complementary site” is meant to describe any region of a gene or gene transcript complementary to the sequence of a let-7 family miRNA, whether or not a let-7 family member can or does bind to that region of the gene or gene transcript in vivo. The term “complementary” describes a threshold of binding between two sequences wherein a majority of nucleotides in each sequence are capable of binding to a majority of nucleotides within the other sequence in trans.

The Human KRAS3′ UTR comprises 8 LCSs named LCS1-LCS8, respectively. For the following sequences, thymine (T) may be substituted for uracil (U). LCS1 comprises the sequence GACAGUGGAAGUUUUUUUUUCCUCG (SEQ ID NO: 1). LCS2 comprises the sequence AUUAGUGUCAUCUUGCCUC (SEQ ID NO: 2). LCS3 comprises the sequence AAUGCCCUACAUCUUAUUUUCCUCA (SEQ ID NO: 3). LCS4 comprises the sequence GGUUCAAGCGAUUCUCGUGCCUCG (SEQ ID NO: 4). LCS5 comprises the sequence GGCUGGUCCGAACUCCUGACCUCA (SEQ ID NO: 5). LCS6 comprises the sequence GAUUCACCCACCUUGGCCUCA (SEQ ID NO: 6). LCS7 comprises the sequence GGGUGUUAAGACUUGACACAGUACCUCG (SEQ ID NO: 7). LCS8 comprises the sequence AGUGCUUAUGAGGGGAUAUUUAGGCCUC (SEQ ID NO: 8).

Human KRAS has two wild type forms, encoded by transcripts a and b, which provided below as SEQ ID NOs: 9 and 10, respectively. The sequences of each human KRAS transcript, containing the LCS6 SNP (KRAS-variant), are provided below as SEQ ID NOs: 11 and 12.

Human KRAS, transcript variant a, is encoded by the following mRNA sequence (NCBI Accession No. NM_033360 and SEQ ID NO: 9) (untranslated regions are bolded, LCS6 is underlined):

```
      1 ggccgcggcg gcggaggcag cagcggcggc ggcagtggcg gcggcgaagg tggcggcggc
    61 tcggccagta ctcccggccc ccgccatttc ggactgggag cgagcgcggc gcaggcactg
  121 aaggcggcgg cggggccaga ggctcagcgg ctcccaggtg cgggagagag gcctgctgaa
  181 aatgactgaa tataaacttg tggtagttgg agctggtggc gtaggcaaga gtgccttgac
  241 gatacagcta attcagaatc attttgtgga cgaatatgat ccaacaatag aggattccta
  301 caggaagcaa gtagtaattg atggagaaac ctgtctcttg gatattctcg acacagcagg
  361 tcaagaggag tacagtgcaa tgagggacca gtacatgagg actggggagg gctttctttg
  421 tgtatttgcc ataaataata ctaaatcatt tgaagatatt caccattata gagaacaaat
  481 taaaagagtt aaggactctg aagatgtacc tatggtccta gtaggaaata aatgtgattt
  541 gccttctaga acagtagaca caaaacaggc tcaggactta gcaagaagtt atggaattcc
  601 ttttattgaa acatcagcaa agacaagaca gagagtggag gatgcttttt atacattggt
  661 gagggagatc cgacaataca gattgaaaaa aatcagcaaa gaagaaaaga ctcctggctg
  721 tgtgaaaatt aaaaaatgca ttataatgta atctgggtgt tgatgatgcc ttctatacat
  781 tagttcgaga aattcgaaaa cataaagaaa agatgagcaa agatggtaaa aagaagaaaa
  841 agaagtcaaa gacaaagtgt gtaattatgt aaatacaatt tgtacttttt tcttaaggca
  901 tactagtaca agtggtaatt tttgtacatt acactaaatt attagcattt gttttagcat
  961 tacctaattt ttttcctgct ccatgcagac tgttagcttt taccttaaat gcttatttta
1021 aaatgacagt ggaagttttt ttttcctcta agtgccagta ttcccagagt tttggttttt
1081 gaactagcaa tgcctgtgaa aaagaaactg aatacctaag atttctgtct tggggttttt
1141 ggtgcatgca gttgattact tcttattttt cttaccaatt gtgaatgttg gtgtgaaaca
1201 aattaatgaa gcttttgaat catccctatt ctgtgtttta tctagtcaca taaatggatt
1261 aattactaat ttcagttgag accttctaat tggtttttac tgaaacattg agggaacaca
1321 aatttatggg cttcctgatg atgattcttc taggcatcat gtcctatagt ttgtcatccc
1381 tgatgaatgt aaagttacac tgttcacaaa ggttttgtct cctttccact gctattagtc
1441 atggtcactc tccccaaaat attatatttt ttctataaaa agaaaaaaat ggaaaaaaat
1501 tacaaggcaa tggaaactat tataaggcca tttccttttc acattagata aattactata
1561 aagactccta atagcttttc ctgttaaggc agacccagta tgaaatgggg attattatag
1621 caaccatttt ggggctatat ttacatgcta ctaaattttt ataataattg aaaagatttt
1681 aacaagtata aaaaattctc ataggaatta aatgtagtct ccctgtgtca gactgctctt
1741 tcatagtata actttaaatc ttttcttcaa cttgagtctt tgaagatagt tttaattctg
1801 cttgtgacat taaaagatta tttgggccag ttatagctta ttaggtgttg aagagaccaa
1861 ggttgcaagg ccaggccctg tgtgaacctt tgagctttca tagagagttt cacagcatgg
1921 actgtgtccc cacggtcatc cagtgttgtc atgcattggt tagtcaaaat ggggagggac
1981 tagggcagtt tggatagctc aacaagatac aatctcactc tgtggtggtc ctgctgacaa
2041 atcaagagca ttgcttttgt ttcttaagaa aacaaactct tttttaaaaa ttacttttaa
2101 atattaactc aaaagttgag attttggggt ggtggtgtgc caagacatta attttttttt
2161 taaacaatga agtgaaaaag ttttacaatc tctaggtttg gctagttctc ttaacactgg
2221 ttaaattaac attgcataaa cacttttcaa gtctgatcca tatttaataa tgctttaaaa
2281 taaaaataaa aacaatcctt ttgataaatt taaaatgtta cttattttaa aataaatgaa
2341 gtgagatggc atggtgaggt gaaagtatca ctggactagg aagaaggtga cttaggttct
2401 agataggtgt cttttaggac tctgattttg aggacatcac ttactatcca tttcttcatg
2461 ttaaaagaag tcatctcaaa ctcttagttt ttttttttta caactatgta atttatattc
2521 catttacata aggatacact tatttgtcaa gctcagcaca atctgtaaat ttttaaccta
2581 tgttacacca tcttcagtgc cagtcttggg caaaattgtg caagaggtga agtttatatt
2641 tgaatatcca ttctcgtttt aggactcttc ttccatatta gtgtcatctt gcctccctac
2701 cttccacatg ccccatgact tgatgcagtt ttaatacttg taattcccct aaccataaga
2761 tttactgctg ctgtggatat ctccatgaag ttttcccact gagtcacatc agaaatgccc
2821 tacatcttat ttcctcaggg ctcaagagaa tctgacagat accataaagg gatttgacct
2881 aatcactaat tttcaggtgg tggctgatgc tttgaacatc tctttgctgc ccaatccatt
2941 agcgacagta ggatttttca aacctggtat gaatagacag aaccctatcc agtggaagga
3001 gaatttaata aagatagtgc tgaaagaatt ccttaggtaa tctataacta ggactactcc
3061 tggtaacagt aatacattcc attgttttag taaccagaaa tcttcatgca atgaaaaata
3121 ctttaattca tgaagcttac tttttttttt tggtgtcaga gtctcgctct tgtcacccag
3181 gctggaatgc agtggcgcca tctcagctca ctgcaacctc catctcccag gttcaagcga
3241 ttctcgtgcc tcggcctcct gagtagctgg gattacaggc gtgtgccact acactcaact
3301 aatttttgta tttttaggag agacggggtt tcaccctgtt ggccaggctg gtctcgaact
3361 cctgacctca agtgattcac ccaccttggc ctcataaacc tgttttgcag aactcattta
3421 ttcagcaaat atttattgag tgcctaccag atgccagtca ccgcacaagg cactgggtat
3481 atggtatccc caaacaagag acataatccc ggtccttagg tagtgctagt gtggtctgta
3541 atatcttact aaggcctttg gtatacgacc cagagataac acgatgcgta ttttagtttt
3601 gcaaagaagg ggtttggtct ctgtgccagc tctataattg ttttgctacg attccactga
3661 aactcttcga tcaagctact ttatgtaaat cacttcattg ttttaaagga ataaacttga
3721 ttatattgtt tttttatttg gcataactgt gattctttta ggacaattac tgtacacatt
3781 aaggtgtatg tcagatattc atattgaccc aaatgtgtaa tattccagtt ttctctgcat
3841 aagtaattaa aatatactta aaaattaata gttttatctg ggtacaaata aacaggtgcc
3901 tgaactagtt cacagacaag gaaacttcta tgtaaaaatc actatgattt ctgaattgct
3961 atgtgaaact acagatcttt ggaacactgt ttaggtaggg tgttaagact tacacagtac
4021 ctcgtttcta cacagagaaa gaaatggcca tacttcagga actgcagtgc ttatgagggg
4081 atatttaggc ctcttgaatt tttgatgtag atgggcattt ttttaaggta gtggttaatt
4141 acctttatgt gaactttgaa tggtttaaca aaagatttgt ttttgtagag attttaaagg
4201 gggagaattc tagaaataaa tgttacctaa ttattacagc cttaaagaca aaaatccttg
4261 ttgaagtttt tttaaaaaaa gctaaattac atagacttag gcattaacat gtttgtggaa
4321 gaatatagca gacgtatatt gtatcatttg agtgaatgtt cccaagtagg cattctaggc
4381 tctatttaac tgagtcacac tgcataggaa tttagaacct aacttttata ggttatcaaa
4441 actgttgtca ccattgcaca attttgtcct aatatataca tagaaacttt gtggggcatg
4501 ttaagttaca gtttgcacaa gttcatctca tttgtattcc attgattttt tttttcttct
4561 aaacattttt tcttcaaaca gtatataact ttttttaggg gatttttttt tagacagcaa
4621 aaactatctg aagatttcca tttgtcaaaa agtaatgatt tcttgataat tgtgtagtaa
4681 tgttttttag aacccagcag ttaccttaaa gctgaattta tatttagtaa cttctgtgtt
4741 aatactggat agcatgaatt ctgcattgag aaactgaata gctgtcataa aatgaaactt
4801 tctttctaaa gaaagatact cacatgagtt cttgaagaat agtcataact agattaagat
4861 ctgtgtttta gtttaatagt ttgaagtgcc tgtttgggat aatgataggt aatttagatg
4921 aatttagggg aaaaaaaagt tatctgcaga tatgttgagg gcccatctct ccccccacac
4981 ccccacagag ctaactgggt tacagtgttt tatccgaaag tttccaattc cactgtcttg
5041 tgttttcatg ttgaaaatac ttttgcattt ttcctttgag tgccaatttc ttactagtac
5101 tatttcttaa tgtaacatgt ttacctggaa tgtattttaa ctatttttgt atagtgtaaa
5161 ctgaaacatg cacattttgt acattgtgct ttcttttgtg ggacatatgc agtgtgatcc
5221 agttgttttc catcatttgg ttgcgctgac ctaggaatgt tggtcatatc aaacattaaa
5281 aatgaccact cttttaattg aaattaactt ttaaatgttt ataggagtat gtgctgtgaa
5341 gtgatctaaa atttgtaata tttttgtcat gaactgtact actcctaatt attgtaatgt
5401 aataaaaata gttacagtga caaaaaaaaa aaaaaa
```

Human KRAS, transcript variant b, is encoded by the following mRNA sequence (NCBI Accession No. NM_004985 and SEQ ID NO: 10) (untranslated regions are bolded, LCS6 is underlined):

```
      1 ggccgcggcg gcggaggcag cagcggcggc ggcagtggcg gcggcgaagg tggcggcggc
    61 tcggccagta ctcccggccc ccgccatttc ggactgggag cgagcgcggc gcaggcactg
  121 aaggcggcgg cggggccaga ggctcagcgg ctcccaggtg cgggagagag gcctgctgaa
  181 aatgactgaa tataaacttg tggtagttgg agctggtggc gtaggcaaga gtgccttgac
  241 gatacagcta attcagaatc attttgtgga cgaatatgat ccaacaatag aggattccta
  301 caggaagcaa gtagtaattg atggagaaac ctgtctcttg gatattctcg acacagcagg
  361 tcaagaggag tacagtgcaa tgagggacca gtacatgagg actggggagg gctttctttg
  421 tgtatttgcc ataaataata ctaaatcatt tgaagatatt caccattata gagaacaaat
  481 taaaagagtt aaggactctg aagatgtacc tatggtccta gtaggaaata aatgtgattt
  541 gccttctaga acagtagaca caaaacaggc tcaggactta gcaagaagtt atggaattcc
  601 ttttattgaa acatcagcaa agacaagaca gggtgttgat gatgccttct atacattagt
  661 tcgagaaatt cgaaaacata aagaaaagat gagcaaagat ggtaaaaaga agaaaaagaa
  721 gtcaaagaca aagtgtgtaa ttatgtaaat acaatttgta cttttttctt aaggcatact
  781 agtacaagtg gtaatttttg tacattacac taaattatta gcatttgttt tagcattacc
  841 taattttttt cctgctccat gcagactgtt agcttttacc ttaaatgctt attttaaaat
  901 gacagtggaa gttttttttt cctctaagtg ccagtattcc cagagttttg gtttttgaac
  961 tagcaatgcc tgtgaaaaag aaactgaata cctaagattt ctgtcttggg gtttttggtg
1021 catgcagttg attacttctt atttttctta ccaattgtga atgttggtgt gaaacaaatt
1081 aatgaagctt ttgaatcatc cctattctgt gttttatcta gtcacataaa tggattaatt
1141 actaatttca gttgagacct tctaattggt ttttactgaa acattgaggg aacacaaatt
1201 tatgggcttc ctgatgatga ttcttctagg catcatgtcc tatagtttgt catccctgat
1261 gaatgtaaag ttacactgtt cacaaaggtt ttgtctcctt tccactgcta ttagtcatgg
1321 tcactctccc caaaatatta tattttttct ataaaaagaa aaaaatggaa aaaaattaca
1381 aggcaatgga aactattata aggccatttc cttttcacat tagataaatt actataaaga
1441 ctcctaatag cttttcctgt taaggcagac ccagtatgaa atggggatta ttatagcaac
1501 cattttgggg ctatatttac atgctactaa atttttataa taattgaaaa gattttaaca
1561 agtataaaaa attctcatag gaattaaatg tagtctccct gtgtcagact gctctttcat
1621 agtataactt taaatctttt cttcaacttg agtctttgaa gatagtttta attctgcttg
1681 tgacattaaa agattatttg ggccagttat agcttattag gtgttgaaga gaccaaggtt
1741 gcaaggccag gccctgtgtg aacctttgag ctttcataga gagtttcaca gcatggactg
1801 tgtccccacg gtcatccagt gttgtcatgc attggttagt caaaatgggg agggactagg
1861 gcagtttgga tagctcaaca agatacaatc tcactctgtg gtggtcctgc tgacaaatca
1921 agagcattgc ttttgtttct taagaaaaca aactcttttt taaaaattac ttttaaatat
1981 taactcaaaa gttgagattt tggggtggtg gtgtgccaag acattaattt tttttttaaa
2041 caatgaagtg aaaaagtttt acaatctcta ggtttggcta gttctcttaa cactggttaa
2101 attaacattg cataaacact tttcaagtct gatccatatt taataatgct ttaaaataaa
2161 aataaaaaca atccttttga taaatttaaa atgttactta ttttaaaata aatgaagtga
2221 gatggcatgg tgaggtgaaa gtatcactgg actaggaaga aggtgactta ggttctagat
2281 aggtgtcttt taggactctg attttgagga catcacttac tatccatttc ttcatgttaa
2341 aagaagtcat ctcaaactct tagttttttt tttttacaac tatgtaattt atattccatt
2401 tacataagga tacacttatt tgtcaagctc agcacaatct gtaaattttt aacctatgtt
2461 acaccatctt cagtgccagt cttgggcaaa attgtgcaag aggtgaagtt tatatttgaa
2521 tatccattct cgttttagga ctcttcttcc atattagtgt catcttgcct ccctaccttc
2581 cacatgcccc atgacttgat gcagttttaa tacttgtaat tcccctaacc ataagattta
2641 ctgctgctgt ggatatctcc atgaagtttt cccactgagt cacatcagaa atgccctaca
2701 tcttatttcc tcagggctca agagaatctg acagatacca taaagggatt tgacctaatc
2761 actaattttc aggtggtggc tgatgctttg aacatctctt tgctgcccaa tccattagcg
2821 acagtaggat ttttcaaacc tggtatgaat agacagaacc ctatccagtg gaaggagaat
2881 ttaataaaga tagtgctgaa agaattcctt aggtaatcta taactaggac tactcctggt
2941 aacagtaata cattccattg ttttagtaac cagaaatctt catgcaatga aaaatacttt
3001 aattcatgaa gcttactttt tttttttggt gtcagagtct cgctcttgtc acccaggctg
3061 gaatgcagtg gcgccatctc agctcactgc aacctccatc tcccaggttc aagcgattct
3121 cgtgcctcgg cctcctgagt agctgggatt acaggcgtgt gccactacac tcaactaatt
3181 tttgtatttt taggagagac ggggtttcac cctgttggcc aggctggtct cgaactcctg
3241 acctcaagtg attcacccac cttggcctca taaacctgtt ttgcagaact catttattca
3301 gcaaatattt attgagtgcc taccagatgc cagtcaccgc acaaggcact gggtatatgg
3361 tatccccaaa caagagacat aatcccggtc cttaggtagt gctagtgtgg tctgtaatat
3421 cttactaagg cctttggtat acgacccaga gataacacga tgcgtatttt agttttgcaa
3481 agaaggggtt tggtctctgt gccagctcta taattgtttt gctacgattc cactgaaact
3541 cttcgatcaa gctactttat gtaaatcact tcattgtttt aaaggaataa acttgattat
3601 attgtttttt tatttggcat aactgtgatt cttttaggac aattactgta cacattaagg
3661 tgtatgtcag atattcatat tgacccaaat gtgtaatatt ccagttttct ctgcataagt
3721 aattaaaata tacttaaaaa ttaatagttt tatctgggta caaataaaca ggtgcctgaa
3781 ctagttcaca gacaaggaaa cttctatgta aaaatcacta tgatttctga attgctatgt
3841 gaaactacag atctttggaa cactgtttag gtagggtgtt aagacttaca cagtacctcg
3901 tttctacaca gagaaagaaa tggccatact tcaggaactg cagtgcttat gaggggatat
3961 ttaggcctct tgaatttttg atgtagatgg gcattttttt aaggtagtgg ttaattacct
4021 ttatgtgaac tttgaatggt ttaacaaaag atttgttttt gtagagattt taaaggggga
4081 gaattctaga aataaatgtt acctaattat tacagcctta aagacaaaaa tccttgttga
4141 agttttttta aaaaaagcta aattacatag acttaggcat taacatgttt gtggaagaat
4201 atagcagacg tatattgtat catttgagtg aatgttccca agtaggcatt ctaggctcta
4261 tttaactgag tcacactgca taggaattta gaacctaact tttataggtt atcaaaactg
4321 ttgtcaccat tgcacaattt tgtcctaata tatacataga aactttgtgg ggcatgttaa
4381 gttacagttt gcacaagttc atctcatttg tattccattg attttttttt tcttctaaac
4441 attttttctt caaacagtat ataacttttt ttaggggatt tttttttaga cagcaaaaac
4501 tatctgaaga tttccatttg tcaaaaagta atgatttctt gataattgtg tagtaatgtt
4561 ttttagaacc cagcagttac cttaaagctg aatttatatt tagtaacttc tgtgttaata
4621 ctggatagca tgaattctgc attgagaaac tgaatagctg tcataaaatg aaactttctt
4681 tctaaagaaa gatactcaca tgagttcttg aagaatagtc ataactagat taagatctgt
4741 gttttagttt aatagtttga agtgcctgtt tgggataatg ataggtaatt tagatgaatt
4801 taggggaaaa aaaagttatc tgcagatatg ttgagggccc atctctcccc ccacaccccc
4861 acagagctaa ctgggttaca gtgttttatc cgaaagtttc caattccact gtcttgtgtt
4921 ttcatgttga aaatactttt gcatttttcc tttgagtgcc aatttcttac tagtactatt
4981 tcttaatgta acatgtttac ctggaatgta ttttaactat ttttgtatag tgtaaactga
5041 aacatgcaca ttttgtacat tgtgctttct tttgtgggac atatgcagtg tgatccagtt
5101 gttttccatc atttggttgc gctgacctag gaatgttggt catatcaaac attaaaaatg
5161 accactcttt taattgaaat taacttttaa atgtttatag gagtatgtgc tgtgaagtga
5221 tctaaaattt gtaatatttt tgtcatgaac tgtactactc ctaattattg taatgtaata
5281 aaaatagtta cagtgacaaa aaaaaaaaaa aa
```

Human KRAS, transcript variant a, comprising the LCS6 SNP (KRAS-variant), is encoded by the following mRNA sequence (SEQ ID NO: 11) (untranslated regions are bolded, LCS6 is underlined, SNP is capitalized):

```
      1 ggccgcggcg gcggaggcag cagcggcggc ggcagtggcg gcggcgaagg tggcggcggc
    61 tcggccagta ctcccggccc ccgccatttc ggactgggag cgagcgcggc gcaggcactg
  121 aaggcggcgg cggggccaga ggctcagcgg ctcccaggtg cgggagagag gcctgctgaa
  181 aatgactgaa tataaacttg tggtagttgg agctggtggc gtaggcaaga gtgccttgac
  241 gatacagcta attcagaatc attttgtgga cgaatatgat ccaacaatag aggattccta
  301 caggaagcaa gtagtaattg atggagaaac ctgtctcttg gatattctcg acacagcagg
  361 tcaagaggag tacagtgcaa tgagggacca gtacatgagg actggggagg gctttctttg
  421 tgtatttgcc ataaataata ctaaatcatt tgaagatatt caccattata gagaacaaat
  481 taaaagagtt aaggactctg aagatgtacc tatggtccta gtaggaaata aatgtgattt
  541 gccttctaga acagtagaca caaaacaggc tcaggactta gcaagaagtt atggaattcc
  601 ttttattgaa acatcagcaa agacaagaca gagagtggag gatgcttttt atacattggt
  661 gagggagatc cgacaataca gattgaaaaa aatcagcaaa gaagaaaaga ctcctggctg
  721 tgtgaaaatt aaaaaatgca ttataatgta atctgggtgt tgatgatgcc ttctatacat
  781 tagttcgaga aattcgaaaa cataaagaaa agatgagcaa agatggtaaa aagaagaaaa
  841 agaagtcaaa gacaaagtgt gtaattatgt aaatacaatt tgtacttttt tcttaaggca
  901 tactagtaca agtggtaatt tttgtacatt acactaaatt attagcattt gttttagcat
  961 tacctaattt ttttcctgct ccatgcagac tgttagcttt taccttaaat gcttatttta
1021 aaatgacagt ggaagttttt ttttcctcta agtgccagta ttcccagagt tttggttttt
1081 gaactagcaa tgcctgtgaa aaagaaactg aatacctaag atttctgtct tggggttttt
1141 ggtgcatgca gttgattact tcttattttt cttaccaatt gtgaatgttg gtgtgaaaca
1201 aattaatgaa gcttttgaat catccctatt ctgtgtttta tctagtcaca taaatggatt
1261 aattactaat ttcagttgag accttctaat tggtttttac tgaaacattg agggaacaca
1321 aatttatggg cttcctgatg atgattcttc taggcatcat gtcctatagt ttgtcatccc
1381 tgatgaatgt aaagttacac tgttcacaaa ggttttgtct cctttccact gctattagtc
1441 atggtcactc tccccaaaat attatatttt ttctataaaa agaaaaaaat ggaaaaaaat
1501 tacaaggcaa tggaaactat tataaggcca tttccttttc acattagata aattactata
1561 aagactccta atagcttttc ctgttaaggc agacccagta tgaaatgggg attattatag
1621 caaccatttt ggggctatat ttacatgcta ctaaattttt ataataattg aaaagatttt
1681 aacaagtata aaaaattctc ataggaatta aatgtagtct ccctgtgtca gactgctctt
1741 tcatagtata actttaaatc ttttcttcaa cttgagtctt tgaagatagt tttaattctg
1801 cttgtgacat taaaagatta tttgggccag ttatagctta ttaggtgttg aagagaccaa
1861 ggttgcaagg ccaggccctg tgtgaacctt tgagctttca tagagagttt cacagcatgg
1921 actgtgtccc cacggtcatc cagtgttgtc atgcattggt tagtcaaaat ggggagggac
1981 tagggcagtt tggatagctc aacaagatac aatctcactc tgtggtggtc ctgctgacaa
2041 atcaagagca ttgcttttgt ttcttaagaa aacaaactct tttttaaaaa ttacttttaa
2101 atattaactc aaaagttgag attttggggt ggtggtgtgc caagacatta attttttttt
2161 taaacaatga agtgaaaaag ttttacaatc tctaggtttg gctagttctc ttaacactgg
2221 ttaaattaac attgcataaa cacttttcaa gtctgatcca tatttaataa tgctttaaaa
2281 taaaaataaa aacaatcctt ttgataaatt taaaatgtta cttattttaa aataaatgaa
2341 gtgagatggc atggtgaggt gaaagtatca ctggactagg aagaaggtga cttaggttct
2401 agataggtgt cttttaggac tctgattttg aggacatcac ttactatcca tttcttcatg
2461 ttaaaagaag tcatctcaaa ctcttagttt ttttttttta caactatgta atttatattc
2521 catttacata aggatacact tatttgtcaa gctcagcaca atctgtaaat ttttaaccta
2581 tgttacacca tcttcagtgc cagtcttggg caaaattgtg caagaggtga agtttatatt
2641 tgaatatcca ttctcgtttt aggactcttc ttccatatta gtgtcatctt gcctccctac
2701 cttccacatg ccccatgact tgatgcagtt ttaatacttg taattcccct aaccataaga
2761 tttactgctg ctgtggatat ctccatgaag ttttcccact gagtcacatc agaaatgccc
2821 tacatcttat ttcctcaggg ctcaagagaa tctgacagat accataaagg gatttgacct
2881 aatcactaat tttcaggtgg tggctgatgc tttgaacatc tctttgctgc ccaatccatt
2941 agcgacagta ggatttttca aacctggtat gaatagacag aaccctatcc agtggaagga
3001 gaatttaata aagatagtgc tgaaagaatt ccttaggtaa tctataacta ggactactcc
3061 tggtaacagt aatacattcc attgttttag taaccagaaa tcttcatgca atgaaaaata
3121 ctttaattca tgaagcttac tttttttttt tggtgtcaga gtctcgctct tgtcacccag
3181 gctggaatgc agtggcgcca tctcagctca ctgcaacctc catctcccag gttcaagcga
3241 ttctcgtgcc tcggcctcct gagtagctgg gattacaggc gtgtgccact acactcaact
3301 aatttttgta tttttaggag agacggggtt tcaccctgtt ggccaggctg gtctcgaact
3361 cctgacctca agtgatGcac ccaccttggc ctcataaacc tgttttgcag aactcattta
3421 ttcagcaaat atttattgag tgcctaccag atgccagtca ccgcacaagg cactgggtat
3481 atggtatccc caaacaagag acataatccc ggtccttagg tagtgctagt gtggtctgta
3541 atatcttact aaggcctttg gtatacgacc cagagataac acgatgcgta ttttagtttt
3601 gcaaagaagg ggtttggtct ctgtgccagc tctataattg ttttgctacg attccactga
3661 aactcttcga tcaagctact ttatgtaaat cacttcattg ttttaaagga ataaacttga
3721 ttatattgtt tttttatttg gcataactgt gattctttta ggacaattac tgtacacatt
3781 aaggtgtatg tcagatattc atattgaccc aaatgtgtaa tattccagtt ttctctgcat
3841 aagtaattaa aatatactta aaaattaata gttttatctg ggtacaaata aacaggtgcc
3901 tgaactagtt cacagacaag gaaacttcta tgtaaaaatc actatgattt ctgaattgct
3961 atgtgaaact acagatcttt ggaacactgt ttaggtaggg tgttaagact tacacagtac
4021 ctcgtttcta cacagagaaa gaaatggcca tacttcagga actgcagtgc ttatgagggg
4081 atatttaggc ctcttgaatt tttgatgtag atgggcattt ttttaaggta gtggttaatt
4141 acctttatgt gaactttgaa tggtttaaca aaagatttgt ttttgtagag attttaaagg
4201 gggagaattc tagaaataaa tgttacctaa ttattacagc cttaaagaca aaaatccttg
4261 ttgaagtttt tttaaaaaaa gctaaattac atagacttag gcattaacat gtttgtggaa
4321 gaatatagca gacgtatatt gtatcatttg agtgaatgtt cccaagtagg cattctaggc
4381 tctatttaac tgagtcacac tgcataggaa tttagaacct aacttttata ggttatcaaa
4441 actgttgtca ccattgcaca attttgtcct aatatataca tagaaacttt gtggggcatg
4501 ttaagttaca gtttgcacaa gttcatctca tttgtattcc attgattttt tttttcttct
4561 aaacattttt tcttcaaaca gtatataact ttttttaggg gatttttttt tagacagcaa
4621 aaactatctg aagatttcca tttgtcaaaa agtaatgatt tcttgataat tgtgtagtaa
4681 tgttttttag aacccagcag ttaccttaaa gctgaattta tatttagtaa cttctgtgtt
4741 aatactggat agcatgaatt ctgcattgag aaactgaata gctgtcataa aatgaaactt
4801 tctttctaaa gaaagatact cacatgagtt cttgaagaat agtcataact agattaagat
4861 ctgtgtttta gtttaatagt ttgaagtgcc tgtttgggat aatgataggt aatttagatg
4921 aatttagggg aaaaaaaagt tatctgcaga tatgttgagg gcccatctct ccccccacac
4981 ccccacagag ctaactgggt tacagtgttt tatccgaaag tttccaattc cactgtcttg
5041 tgttttcatg ttgaaaatac ttttgcattt ttcctttgag tgccaatttc ttactagtac
5101 tatttcttaa tgtaacatgt ttacctggaa tgtattttaa ctatttttgt atagtgtaaa
5161 ctgaaacatg cacattttgt acattgtgct ttcttttgtg ggacatatgc agtgtgatcc
5221 agttgttttc catcatttgg ttgcgctgac ctaggaatgt tggtcatatc aaacattaaa
5281 aatgaccact cttttaattg aaattaactt ttaaatgttt ataggagtat gtgctgtgaa
5341 gtgatctaaa atttgtaata tttttgtcat gaactgtact actcctaatt attgtaatgt
5401 aataaaaata gttacagtga caaaaaaaaa aaaaaa
```

Human KRAS, transcript variant b, comprising the LCS6 SNP (KRAS-variant), is encoded by the following mRNA sequence (SEQ ID NO: 12) (untranslated regions are bolded, LCS6 is underlined, SNP is capitalized):

```
      1 ggccgcggcg gcggaggcag cagcggcggc ggcagtggcg gcggcgaagg tggcggcggc
    61 tcggccagta ctcccggccc ccgccatttc ggactgggag cgagcgcggc gcaggcactg
  121 aaggcggcgg cggggccaga ggctcagcgg ctcccaggtg cgggagagag gcctgctgaa
  181 aatgactgaa tataaacttg tggtagttgg agctggtggc gtaggcaaga gtgccttgac
  241 gatacagcta attcagaatc attttgtgga cgaatatgat ccaacaatag aggattccta
  301 caggaagcaa gtagtaattg atggagaaac ctgtctcttg gatattctcg acacagcagg
  361 tcaagaggag tacagtgcaa tgagggacca gtacatgagg actggggagg gctttctttg
  421 tgtatttgcc ataaataata ctaaatcatt tgaagatatt caccattata gagaacaaat
  481 taaaagagtt aaggactctg aagatgtacc tatggtccta gtaggaaata aatgtgattt
  541 gccttctaga acagtagaca caaaacaggc tcaggactta gcaagaagtt atggaattcc
  601 ttttattgaa acatcagcaa agacaagaca gggtgttgat gatgccttct atacattagt
  661 tcgagaaatt cgaaaacata aagaaaagat gagcaaagat ggtaaaaaga agaaaaagaa
  721 gtcaaagaca aagtgtgtaa ttatgtaaat acaatttgta cttttttctt aaggcatact
  781 agtacaagtg gtaatttttg tacattacac taaattatta gcatttgttt tagcattacc
  841 taattttttt cctgctccat gcagactgtt agcttttacc ttaaatgctt attttaaaat
  901 gacagtggaa gttttttttt cctctaagtg ccagtattcc cagagttttg gtttttgaac
  961 tagcaatgcc tgtgaaaaag aaactgaata cctaagattt ctgtcttggg gtttttggtg
1021 catgcagttg attacttctt atttttctta ccaattgtga atgttggtgt gaaacaaatt
1081 aatgaagctt ttgaatcatc cctattctgt gttttatcta gtcacataaa tggattaatt
1141 actaatttca gttgagacct tctaattggt ttttactgaa acattgaggg aacacaaatt
1201 tatgggcttc ctgatgatga ttcttctagg catcatgtcc tatagtttgt catccctgat
1261 gaatgtaaag ttacactgtt cacaaaggtt ttgtctcctt tccactgcta ttagtcatgg
1321 tcactctccc caaaatatta tattttttct ataaaaagaa aaaaatggaa aaaaattaca
1381 aggcaatgga aactattata aggccatttc cttttcacat tagataaatt actataaaga
1441 ctcctaatag cttttcctgt taaggcagac ccagtatgaa atggggatta ttatagcaac
1501 cattttgggg ctatatttac atgctactaa atttttataa taattgaaaa gattttaaca
1561 agtataaaaa attctcatag gaattaaatg tagtctccct gtgtcagact gctctttcat
1621 agtataactt taaatctttt cttcaacttg agtctttgaa gatagtttta attctgcttg
1681 tgacattaaa agattatttg ggccagttat agcttattag gtgttgaaga gaccaaggtt
1741 gcaaggccag gccctgtgtg aacctttgag ctttcataga gagtttcaca gcatggactg
1801 tgtccccacg gtcatccagt gttgtcatgc attggttagt caaaatgggg agggactagg
1861 gcagtttgga tagctcaaca agatacaatc tcactctgtg gtggtcctgc tgacaaatca
1921 agagcattgc ttttgtttct taagaaaaca aactcttttt taaaaattac ttttaaatat
1981 taactcaaaa gttgagattt tggggtggtg gtgtgccaag acattaattt tttttttaaa
2041 caatgaagtg aaaaagtttt acaatctcta ggtttggcta gttctcttaa cactggttaa
2101 attaacattg cataaacact tttcaagtct gatccatatt taataatgct ttaaaataaa
2161 aataaaaaca atccttttga taaatttaaa atgttactta ttttaaaata aatgaagtga
2221 gatggcatgg tgaggtgaaa gtatcactgg actaggaaga aggtgactta ggttctagat
2281 aggtgtcttt taggactctg attttgagga catcacttac tatccatttc ttcatgttaa
2341 aagaagtcat ctcaaactct tagttttttt tttttacaac tatgtaattt atattccatt
2401 tacataagga tacacttatt tgtcaagctc agcacaatct gtaaattttt aacctatgtt
2461 acaccatctt cagtgccagt cttgggcaaa attgtgcaag aggtgaagtt tatatttgaa
2521 tatccattct cgttttagga ctcttcttcc atattagtgt catcttgcct ccctaccttc
2581 cacatgcccc atgacttgat gcagttttaa tacttgtaat tcccctaacc ataagattta
2641 ctgctgctgt ggatatctcc atgaagtttt cccactgagt cacatcagaa atgccctaca
2701 tcttatttcc tcagggctca agagaatctg acagatacca taaagggatt tgacctaatc
2761 actaattttc aggtggtggc tgatgctttg aacatctctt tgctgcccaa tccattagcg
2821 acagtaggat ttttcaaacc tggtatgaat agacagaacc ctatccagtg gaaggagaat
2881 ttaataaaga tagtgctgaa agaattcctt aggtaatcta taactaggac tactcctggt
2941 aacagtaata cattccattg ttttagtaac cagaaatctt catgcaatga aaaatacttt
3001 aattcatgaa gcttactttt tttttttggt gtcagagtct cgctcttgtc acccaggctg
3061 gaatgcagtg gcgccatctc agctcactgc aacctccatc tcccaggttc aagcgattct
3121 cgtgcctcgg cctcctgagt agctgggatt acaggcgtgt gccactacac tcaactaatt
3181 tttgtatttt taggagagac ggggtttcac cctgttggcc aggctggtct cgaactcctg
3241 acctcaagtg atGcacccac cttggcctca taaacctgtt ttgcagaact catttattca
3301 gcaaatattt attgagtgcc taccagatgc cagtcaccgc acaaggcact gggtatatgg
3361 tatccccaaa caagagacat aatcccggtc cttaggtagt gctagtgtgg tctgtaatat
3421 cttactaagg cctttggtat acgacccaga gataacacga tgcgtatttt agttttgcaa
3481 agaaggggtt tggtctctgt gccagctcta taattgtttt gctacgattc cactgaaact
3541 cttcgatcaa gctactttat gtaaatcact tcattgtttt aaaggaataa acttgattat
3601 attgtttttt tatttggcat aactgtgatt cttttaggac aattactgta cacattaagg
3661 tgtatgtcag atattcatat tgacccaaat gtgtaatatt ccagttttct ctgcataagt
3721 aattaaaata tacttaaaaa ttaatagttt tatctgggta caaataaaca ggtgcctgaa
3781 ctagttcaca gacaaggaaa cttctatgta aaaatcacta tgatttctga attgctatgt
3841 gaaactacag atctttggaa cactgtttag gtagggtgtt aagacttaca cagtacctcg
3901 tttctacaca gagaaagaaa tggccatact tcaggaactg cagtgcttat gaggggatat
3961 ttaggcctct tgaatttttg atgtagatgg gcattttttt aaggtagtgg ttaattacct
4021 ttatgtgaac tttgaatggt ttaacaaaag atttgttttt gtagagattt taaaggggga
4081 gaattctaga aataaatgtt acctaattat tacagcctta aagacaaaaa tccttgttga
4141 agttttttta aaaaaagcta aattacatag acttaggcat taacatgttt gtggaagaat
4201 atagcagacg tatattgtat catttgagtg aatgttccca agtaggcatt ctaggctcta
4261 tttaactgag tcacactgca taggaattta gaacctaact tttataggtt atcaaaactg
4321 ttgtcaccat tgcacaattt tgtcctaata tatacataga aactttgtgg ggcatgttaa
4381 gttacagttt gcacaagttc atctcatttg tattccattg attttttttt tcttctaaac
4441 attttttctt caaacagtat ataacttttt ttaggggatt tttttttaga cagcaaaaac
4501 tatctgaaga tttccatttg tcaaaaagta atgatttctt gataattgtg tagtaatgtt
4561 ttttagaacc cagcagttac cttaaagctg aatttatatt tagtaacttc tgtgttaata
4621 ctggatagca tgaattctgc attgagaaac tgaatagctg tcataaaatg aaactttctt
4681 tctaaagaaa gatactcaca tgagttcttg aagaatagtc ataactagat taagatctgt
4741 gttttagttt aatagtttga agtgcctgtt tgggataatg ataggtaatt tagatgaatt
4801 taggggaaaa aaaagttatc tgcagatatg ttgagggccc atctctcccc ccacaccccc
4861 acagagctaa ctgggttaca gtgttttatc cgaaagtttc caattccact gtcttgtgtt
4921 ttcatgttga aaatactttt gcatttttcc tttgagtgcc aatttcttac tagtactatt
4981 tcttaatgta acatgtttac ctggaatgta ttttaactat ttttgtatag tgtaaactga
5041 aacatgcaca ttttgtacat tgtgctttct tttgtgggac atatgcagtg tgatccagtt
5101 gttttccatc atttggttgc gctgacctag gaatgttggt catatcaaac attaaaaatg
5161 accactcttt taattgaaat taacttttaa atgtttatag gagtatgtgc tgtgaagtga
5221 tctaaaattt gtaatatttt tgtcatgaac tgtactactc ctaattattg taatgtaata
5281 aaaatagtta cagtgacaaa aaaaaaaaaa aa
```

The present invention encompasses a SNP within the 3′UTR of KRAS. Specifically, this SNP is the result of a substitution of a G in place of U at position 4 of SEQ ID NO: 6 of LCS6. This LCS6 SNP (KRAS-variant) comprises the sequence GAUGCACCCACCUUGGCCUCA (SNP bolded for emphasis) (SEQ ID NO: 13).

The KRAS-variant leads to altered KRAS expression by disrupting the miRNA regulation of a KRAS. The identification and characterization of the KRAS-variant is further described in International Application No. PCT/US08/65302 (WO 2008/151004), the contents of which are incorporated by reference in its entirety.

### Methods of Treating Cancer

The present inventors discovered that the presence of the KRAS-variant increases the relative likelihood of responding to administration of one specific type of immune modulator than another in a cancer patient. Specifically, the present invention is directed to methods of selecting a specific immune modulating agent to be administered to a patient in need thereof, wherein the choice of the immune modulating agent to be administered is dependent on the presence of a KRAS-variant. In the presence of the KRAS-variant, immune modulating agents that function to initially stimulate a weakened immune system are preferred over agents which rely on a fully functional immune system for their benefit. Immune modulating agents to be administered to KRAS-variant patients include, for example, antibodies, cytokines, adoptive cell transfer, while those agents such as checkpoint inhibitors are less preferred.

Accordingly, the present invention relates to a method of administering an immune modulator to a cancer patient in need thereof wherein said administration is dependent on the presence of the KRAS-variant in said patient. In an embodiment of the invention, the method may include administering to the KRAS-variant subject an immune modulator in combination with another cancer treatment such as surgery, chemotherapy or radiation therapy. In a preferred embodiment, an immune modulator is administered in conjunction with radiation therapy.

The present inventors also discovered that the presence of the KRAS-variant reduces the likelihood of a cancer patient having a toxic response to an immunotherapy. Specifically, the present invention is directed to reduced-toxicity methods of treating cancer, where an immune modulator is administered to a patient in need thereof, wherein administration of the immune modulator is dependent on the presence of a KRAS-variant. In another embodiment, the invention is directed to a method of predicting the toxicity of an immune modulator in a patient, where the method requires detecting the presence of a KRAS-variant and the immune modulator is administered to a patient if the KRAS-variant is detected, as the presence of the KRAS-variant indicates a reduced likelihood of toxicity of the immune modulator in the subject.

As used herein, the term “immunotherapy” relates to any immune-based therapy designed to stimulate the immune system for inhibition, or destruction, of cancer cells. In one aspect of the invention, the immunotherapy comprises administration of an immune modulating agent that enhances innate as well as adaptive immunity in a patient.

As used herein, the term “toxicity” or “toxic response” refers to the occurrence of one or more immune response adverse reaction(s) (irAEs), a particular class of adverse reactions a patient may experience in response to a cancer therapy, and most commonly cancer immunotherapy. irAEs are believed to occur as a result of stimulation of the immune system by the cancer therapy and include different forms of auto-immunity induced by the administration of these therapies, such as, for example, pneumonitis, hepatitis, pancreatitis, and colitis. For example, irAEs are particularly observed in patients who are treated with checkpoint inhibitor therapies. irAEs are more fully discussed in, for example, Abdel-Wahab et al., PLos ONE, 11(7):e0160221 (2016).

In one aspect of the invention, an immunoglobulin molecule, or fragment thereof, designed to recognize and target destruction of cancer cells may be administered. Such immunoglobulin molecules include, for example, monoclonal antibodies such as cetuximab, panitumumab, bevacizumab, rituximab and trastuzumab. Antibodies that recognize VEGF such as for example Avastin may also be used. In addition to targeting antigens involved in cancer cell physiology, administered antibodies may also function to modulate immunological pathways that are critical to immune surveillance.

In another aspect of the invention, administration of chemotherapeutic agents known to stimulate, or which rely on, the immune system may be particularly useful, or not useful to treat those cancer patients having the KRAS-variant. Such agents include, but are not limited to, erlotinib, vandetanib, cisplatin, irinotecan, etoposide, taxol, raf inhibitors such as sorafenib, celecoxib, cetuximab and panitumimab. The combination of agents and their impact on the immune system is critical for KRAS-variant patients, with certain combinations, that together enhance immunity, being useful, and other combinations, that may hinder immunity, being harmful or non-useful. Such agents may have, for example, one or more of the following immunostimulatory properties: enhancement of cancer cell susceptibility to NK (natural killer) and/or cytotoxic T lymphocyte (CTL) mediated cell lysis, stimulation of mature dendritic cell (DC) and CD8 T-cell numbers, a decrease in immunosuppression by DC and tumor cells, induced activation of DC, NK and tumor specific CTLs, augmentation ofThl cellular immunity, stimulation of TYRO3, AXL and MER (TAM) receptor protein tyrosine kinase mediated cytotoxicity, enhancement of expression of cancer cell antigens enabling recognition by T-lymphocytes, enhancement of antibody-dependent cell-mediated cytotoxicity (ADCC) and complement dependent cytotoxicity (CDC) and general enhancement of innate and adoptive immunity.

Alternatively, immunostimulatory molecules such as cytokines that function to activate cells of the immune system may be administered. Such factors include, for example, T-cell activators or a dendritic cell activation/maturation factors. Additionally, adoptive cell transfer may be used wherein T-cells that have a natural or genetically engineered reactivity to a patient's cancer are generated in vitro and then transferred back into the cancer patient. In addition, the patients T-cells may be removed and genetically engineered to express a T-cell receptor gene (TCR) gene that is specialized to recognize tumor antigens. The cells are then transferred back into the patient for targeted destruction of the cancer cells.

In another aspect, the invention further provides that KRAS-variant cancer subjects may respond better to one immunotherapy versus another at specific times during a particular treatment protocol. For example, checkpoint inhibitors that function downstream of a stimulated immune system may be administered following initial immune system stimulation. Checkpoint inhibitors normally acts as a type of “off switch” that helps keep the T cells from attacking other cells in the body, including cancer cells. In some instances, checkpoint inhibitors may be administered to the cancer subject to remove the “off switch” thereby enhancing the cancer subjects T-cell response against cancer cells. Such checkpoint inhibitors include, for example, treatments that target and inhibit CTLA-4, PD-1 or PD-L1, boosting the immune response against cancer cells. Examples of treatments that target PD-1 include Pembrolizumab (Keytruda®) and Nivolumab (Opdivo®). Examples of treatments that target PD-L1 are BMS-936559 (MDX-1105), Tecentriq® (atezolizumab), durvalumab (MEDI4736), and Bavencio® (avelumab). Ipilimumab (Yervoy®) is a monoclonal antibody that targets CTLA-4 and prevents the protein from inhibiting cytotoxic T lymphocytes. This can boost the body's immune response against cancer cells.

In addition, the present invention provides a means for identification of a suitable target patient, or target subpopulation of patients, for clinical trial design. Accordingly, subjects having the KRAS-variant may be chosen for clinical trials wherein said treatment involves administration of a drug, or treatment, designed to stimulate, or enhance, the immune system, while such subjects would be excluded from trials involving checkpoint inhibitors. Alternatively, subjects having the KRAS-variant may be chosen for clinical trials wherein the efficacy of a test drug is enhanced by co-administration of an immunotherapy. Such a targeted selection of test subjects may serve to streamline the drug approval process by reducing the size and numbers of trials thereby facilitating quick regulatory approval and advancement of the drug to market.

In instances where it is found that the presence of the KRAS-variant is associated with increased efficacy of a tested drug, the present invention further provides methods for testing of a patient for the presence of the KRAS-variant prior to prescribing of the tested/approved drug by a physician. In such instances, the drug label may contain instructions that the patient should be tested for presence of the KRAS-variant prior to administration of the drug. Accordingly, the present invention is also directed to a combination drug label wherein said label refers to the use of a drug which as a condition of use must be used in combination with a diagnostic test wherein said diagnostic test is designed to detect the presence of a KRAS-variant in said subject. More specifically, the invention provides diagnostic methods for testing of a patient prior to prescribing of a drug, and to combination drug labels, wherein the diagnostic test comprises detecting a single nucleotide polymorphism (SNP) at position 4 of the let-7 complementary site 6 of KRAS in a patient sample wherein the presence of said SNP indicates an increased beneficial effect resulting from immunotherapy.

“Treat” as used herein refers to any type of treatment or prevention that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc. As such, the term “treatment” also includes prophylactic treatment of the subject to prevent the onset of symptoms.

As used herein, “treatment” and “prevention” are not meant to imply cure or complete abatement of symptoms. Rather, these refer to any type of treatment that imparts a benefit to a patient afflicted with a disease, including improvement in the condition of the patient (e.g., in one or more symptoms), delay in the progression of the disease, etc.

“Treatment-effective amount” as used herein means an amount of the immunotherapy sufficient to produce a desirable effect upon a patient inflicted with cancer, including improvement in the condition of the patient (e.g., in one or more symptoms), delay in the progression of the disease, etc.

Subjects in need of treatment by the methods described herein include subjects afflicted with tumors and cancers such as, for example, lung, colon, breast, brain, liver, prostate, spleen, muscle, ovary, pancreas, head and neck, skin (including melanoma), etc. The tumor may be a primary tumor, a metastatic tumor, or a recurrent tumor.

The terms “therapeutic agent”, “chemotherapeutic agent”, or “drug” as used herein refers to a compound or a derivative thereof that can interact with a cancer cell, thereby reducing the proliferative status of the cell and/or killing the cell. Examples of chemotherapeutic agents include, but are not limited to, alkylating agents (e.g., cyclophosphamide, ifosamide), metabolic antagonists (e.g., methotrexate (MTX), 5-fluorouracil or derivatives thereof), antitumor antibiotics (e.g., mitomycin, adriamycin), plant-derived antitumor agents (e.g., vincristine, vindesine, Taxol), platinum-based chemotherapeutics (e.g., cisplatin, carboplatin, oxaliplatin, nedaplatin, triplatin tetranitrate, phenanthriplatin, picoplatin, satraplatin), etoposide, and the like. Such agents may further include, but are not limited to, the anti-cancer agents trimethotrixate (TMTX), temozolomide, realtritrexed, S-(4-Nitrobenzyl)-6-thioinosine (NBMPR), 6-benzyguanidine (6-BG), bis-chloronitrosourea (BCNU) and camptothecin, or a therapeutic derivative of any thereof.

The term “radiation therapy” as used herein refers to radiation therapies that use high-energy radiation to shrink tumors and kill cancer cells. X-rays, gamma rays, photons and charged particles are types of radiation used for cancer treatment. The radiation may be delivered from outside the body (external-beam radiation therapy), or it may be delivered by placement of radioactive material in the body near cancer cells (internal radiation therapy, also called brachytherapy). Systemic radiation therapy uses radioactive substances, such as radioactive iodine, that travel in the blood to kill cancer cells. As described herein, KRAS-variant patients are observed to have an increased sensitivity to radiation therapy in their normal tissues, but, fail of distant disease due to their baseline immunosuppression, indicating their need for immune enhancement.

The term “therapeutically effective amount” as used herein refers to that amount of the compound being administered that will relieve to some extent one or more of the symptoms of a disease, a condition, or a disorder being treated. In reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

The terms “treating” or “treatment” of a disease (or a condition or a disorder) as used herein refer to preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer, these terms also mean that the life expectancy of an individual affected with a cancer may be increased or that one or more of the symptoms of the disease will be reduced.

The terms “subject” and “patient” as used herein include humans, mammals (e.g., cats, dogs, horses, etc.), living cells, and other living organisms.

The term “cancer,” as used herein, shall be given its ordinary meaning, as a general term for diseases in which abnormal cells divide without control. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (although some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

### Formulations

Pharmaceutical compositions of the disclosure (e.g., chemotherapeutics and/or immunotherapeutics) may be administered by various means, depending on their intended use, as is well known in the art. For example, if compositions of the disclosure are to be administered orally, they may be formulated as tablets, capsules, granules, powders or syrups. Alternatively, formulations disclosed herein may be administered parenterally as injections (intravenous, intramuscular or subcutaneous), drop infusion preparations or suppositories. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent. The disclosed excipients may serve more than one function. For example, fillers or binders may also be disintegrants, glidants, anti-adherents, lubricants, sweeteners and the like.

In formulations of the disclosure, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal (e.g., by inhalation using a dry powder formulation or a nebulized formulation), topical (including buccal and sublingual), pulmonary (including aerosol administration), rectal, vaginal, aerosol and/or parenteral (e.g., by injection, for example, intravenous or subcutaneous injection) administration. The formulations may conveniently be presented in unit dosage form and may be prepared by any methods well known in the art of pharmacy. The amounts of a composition that may be combined with a carrier material to produce a single dose vary depending upon the subject being treated, and the particular mode of administration.

Methods of preparing these formulations include the step of bringing into association compositions of the disclosure with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the disclosure may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, dextrose, lactose, sucrose, glucose, mannitol, and/or silicic acid; (2) binders, such as, for example, celluloses (e.g., microcrystalline cellulose, methyl cellulose, hydroxypropylmethyl cellulose (HPMC) and carboxymethylcellulose), alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like. The disclosed excipients may serve more than one function. For example, fillers or binders may also be disintegrants, glidants, anti-adherents, lubricants, sweeteners and the like.

Formulations and compositions may include micronized crystals of the disclosed compounds. Micronization may be performed on crystals of the compounds alone, or on a mixture of crystals and a part or whole of pharmaceutical excipients or carriers. Mean particle size of micronized crystals of a disclosed compound may be for example about 5 to about 200 microns, or about 10 to about 110 microns.

A tablet may be made by compression or molding, optionally with one or more accessory ingredients. Compressed tablets may be prepared using binder (for example, gelatin, microcrystalline cellulose, or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art. The disclosed excipients may serve more than one function. For example, fillers or binders may also be disintegrants, glidants, anti-adherents, lubricants, sweeteners and the like.

It will be appreciated that a disclosed composition may include lyophilized or freeze dried compounds disclosed herein. For example, disclosed herein are compositions that disclosed compounds crystalline and/or amorphous powder forms. Such forms may be reconstituted for use as e.g., an aqueous composition.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, com, germ, olive, castor and sesame oils), glycerol, tetrahydrofuyl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, and patches. The active component may be mixed under sterile conditions with a pharmaceutically acceptable carrier, and with any preservatives, buffers, or propellants that may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

It should be noted that excipients given as examples may have more than one function. For example, fillers or binders can also be disintegrants, glidants, anti-adherents, lubricants, sweeteners and the like.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. For example, provided herein is an aqueous composition that includes a disclosed compound, and may further include for example, dextrose (e.g., about 1 to about 10 weight percent dextrose, or about 5 weight percent dextrose in water (D5W).

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

It will be appreciated that contemplated formulations, such as oral formulations (e.g. a pill or tablet), may be formulated as controlled release formulation, e.g., an immediate release formulation, a delayed release formulation, or a combination thereof.

In certain embodiments, the subject compounds may be formulated as a tablet, pill, capsule or other appropriate ingestible formulation (collectively hereinafter “tablet”). In certain embodiments, a therapeutic dose may be provided in 10 tablets or fewer. In another example, a therapeutic dose is provided in 50, 40, 30, 20, 15, 10, 5 or 3 tablets.

For purposes of the invention, the amount or dose of the immune modulating agent should be sufficient to effect, e.g., a therapeutic or prophylactic response, in the subject or animal over a reasonable time frame. For example, the dose of the immune modulating agent should be sufficient to bind to a cancer antigen, or detect, treat or prevent cancer in a subject. The dose will be determined by the efficacy of the agent and the condition of the subject (e.g., human), as well as the body weight of the subject (e.g., human) to be treated. Assays for determining an administered dosages are well known in the art.

The dose of the immune modulating agent containing composition can also be determined by the existence, nature and extent of any adverse side effects that might accompany the administration of a particular agent. Typically, the attending physician will decide the dosage of the agent with which to treat each individual patient, taking into consideration a variety of factors, such as age, body weight, general health, diet, sex, route of administration, and the severity of the condition being treated.

### Administration of Immunotherapies

Immunotherapies described may be antibody based therapies. Generally, a therapeutically effective amount of the antibody is in the range of 0.1 mg/kg to 100 mg/kg, e.g., 1 mg/kg to 100 mg/kg, e.g., 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. The optimal dose can be determined by routine experimentation. For parenteral administration a dose between 0.1 mg/kg and 100 mg/kg, alternatively between 0.5 mg/kg and 50 mg/kg, alternatively, between 1 mg/kg and 25 mg/kg, alternatively between 2 mg/kg and 10 mg/kg, alternatively between 5 mg/kg and 10 mg/kg is administered and may be given, for example, once weekly, once every other week, once every third week, or once monthly per treatment cycle. In one embodiment, the dose is 200 mg every 3 weeks via intravenous administration, whereas in another embodiment, the dose is 2 mg/kg every 3 weeks via intravenous administration. In another embodiment, the dose is 240 mg every 2 weeks via intravenous administration, while in yet another embodiment, the dose is or 3 mg/kg every 2 weeks via intravenous administration. In yet another embodiment, the dose is 1200 mg every 3 weeks via intravenous administration.

### Methods of Predicting Likelihood of Responding to Immunotherapy or Likelihood of Toxic Response to Immunotherapy

The invention also features methods of predicting an increased likelihood of responding to immunotherapy, either alone, or in combination with one or more conventional cancer treatments. The method includes detecting a single nucleotide polymorphism (SNP) at position 4 of the let-7 complementary site 6 of KRAS in a patient sample wherein the presence of said SNP indicates an increased likelihood of responding to immunotherapy in a cancer subject. Specifically the mutation that is detected is a SNP at position 4 of LCS6 of KRAS of which results in a uracil (U) or thymine (T) to guanine (G) conversion. In certain non-limiting embodiments, the cancer is breast cancer, ovarian cancer, non-small cell lung cancer, colorectal cancer, melanoma, or head and neck cancer. Identification of the mutation indicates an increased likelihood of responding to immunotherapy.

An “increased likelihood” is meant to describe an increased probability that an individual who carries the KRAS-variant responds to immunotherapy, compared to an individual who does not carry the KRAS-variant. In certain embodiments, a KRAS-variant carrier is 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, or 100× more likely to respond to immunotherapy than an individual who does not carry the KRAS-variant.

A subject is preferably a mammal. The mammal can be a human, non-human primate, mouse, rat, dog, cat, horse, or cow, but are not limited to these examples. A subject can be male or female.

The invention also features methods of predicting a reduced likelihood of having a toxic response to immunotherapy. The method includes detecting a single nucleotide polymorphism (SNP) at position 4 of the let-7 complementary site 6 of KRAS in a patient sample wherein the presence of said SNP indicates a reduced likelihood of the patient having a toxic response to immunotherapy. Specifically the mutation that is detected is a SNP at position 4 of LCS6 of KRAS of which results in a uracil (U) or thymine (T) to guanine (G) conversion. In certain non-limiting embodiments, the cancer is breast cancer, ovarian cancer, non-small cell lung cancer, colorectal cancer, melanoma, or head and neck cancer. Identification of the mutation indicates a reduced likelihood of having a toxic response to immunotherapy.

A “reduced likelihood” is meant to describe a reduced probability that an individual who carries the KRAS-variant has a toxic response to immunotherapy, compared to an individual who does not carry the KRAS-variant. In certain embodiments, a KRAS-variant carrier is 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 20×, 30×, 40×, 50×, 60×, 70×, 80×, 90×, or 100× less likely to have a toxic response to immunotherapy than an individual who does not carry the KRAS-variant.

“Likelihood” in the context of the present invention, relates to the probability that an event will occur over a specific time period, and can mean a subject's “absolute” likelihood or “relative” likelihood. Absolute likelihood can be measured with reference to either actual observation post-measurement for the relevant time cohort, or with reference to index values developed from statistically valid historical cohorts that have been followed for the relevant time period. Relative likelihood refers to the ratio of absolute likelihoods of a subject compared either to the absolute likelihoods of low likelihood cohorts or an average population likelihood, which can vary by how clinical likelihood factors are assessed. Odds ratios, the proportion of positive events to negative events for a given test result, are also commonly used (odds are according to the formula p/(1−p) where p is the probability of event and (1−p) is the probability of no event) to no-conversion.

“Likelihood evaluation” or “evaluation of likelihood” in the context of the present invention encompasses making a prediction of the probability, odds, or likelihood that a cancer subject will respond to immunotherapy or have a toxic response to immunotherapy. Such an evaluation can also comprise prediction of future clinical parameters, traditional laboratory risk factor values, or other indices of cancer, either in absolute or relative terms in reference to a previously measured population.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium.” In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For screening individuals for genetic disorders (e.g. prognostic or risk) purposes, if a particular SNP site is found to be useful for screening a disorder, then the skilled artisan would recognize that other SNP sites which are in LD with this SNP site would also be useful for screening the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For screening applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., disease) that is influenced by the causative SNP(s). Thus, polymorphic markers that are in LD with causative polymorphisms are useful as markers, and are particularly useful when the actual causative polymorphism(s) is/are unknown.

Linkage disequilibrium in the human genome is reviewed in: Wall et al. (2003) NAT REV GENET. 4(8):587-97; Gamer et al. (2003) GENET EPIDEMIOL. 24 (1):57-67; Ardlie et al. (2002) NAT REV GENET. 3(4):299-309 (erratum in (2002) NAT REV GENET 3(7):566); and Remm et al. (2002) CURR OPIN CHEM BIOL. 6(1):24-30.

The screening techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders.

### SNP Genotyping Methods

The process of determining which specific nucleotide (i.e., allele) is present at each of one or more SNP positions, such as a SNP position in a nucleic acid molecule disclosed in SEQ ID NO: 11, 12 or 13, is referred to as SNP genotyping. The present invention provides methods of SNP genotyping, such as for use in screening for a variety of disorders, or determining predisposition thereto, or determining responsiveness to a form of treatment, or prognosis, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al. (2003) PHARMACOGENOMICS J. 3(2):77-%; Kwok et al. (2003) CURR ISSUES MOL. BIOL. 5(2):43-60; Shi (2002) AM J PHARMACOGENOMICS 2(3):197-205; and Kwok (2001) ANNU REV GENOMICS HUM GENET 2:235-58. Exemplary techniques for high-throughput SNP genotyping are described in Mamellos (2003) CURR OPIN DRUG DISCOV DEVEL. 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

Various methods for detecting polymorphisms include, but are not limited to, methods in which protection from cleavage agents is used to detect mismatched bases in RNA/RNA or RNA/DNA duplexes (Myers et al. (1985) SCIENCE 230: 1242; Cotton et al. (1988) PNAS 85:4397; and Saleeba et al. (1992) METH. ENZYMOL. 217:286-295), comparison of the electrophoretic mobility of variant and wild type nucleic acid molecules (Orita et al. (1989) PNAS 86:2766; Cotton et al. (1993) MUTAT. RES. 285:125-144; and Hayashi et al. (1992) GENET. ANAL. TECH. APPL. 9:73-79), and assaying the movement of polymorphic or wild-type fragments in polyacrylamide gels containing a gradient of denaturant using denaturing gradient gel electrophoresis (DGGE) (Myers et al. (1985), NATURE 313 :495). Sequence variations at specific locations can also be assessed by nuclease protection assays such as RNase and SI protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in prognostic assays for a variety of disorders including cancer, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

The identity of polymorphisms may also be determined using a mismatch detection technique, including but not limited to the RNase protection method using riboprobes (Winter et al. (1985) PNAS 82:7575; Meyers et al. (1985) Science 230: 1242) and proteins which recognize nucleotide mismatches, such as the E. coli mutS protein (Modrich (1991) Ann. Rev. Genet. 25:229-253). Alternatively, variant alleles can be identified by single strand conformation polymorphism (SSCP) analysis (Orita et al. (1989) Genomics 5:874-879; Humphries et al., in Molecular Diagnosis of Genetic Diseases, R. Elles, ed., pp. 321-340, 1996) or denaturing gradient gel electrophoresis (DGGE) (Wartell et al. (1990) Nucl. Acids Res. 18:2699-2706; Sheffield et al. (1989) PNAS 86:232-236).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruano et al. (1989) NUCL. ACIDS RES. 17:8392; Ruano et al. (1991) NUCL. ACIDS RES. 19, 6877-6882; WO 93/22456; Turki et al. (1995) J CLIN. INVEST. 95: 1635-1641). In addition, multiple polymorphic sites may be investigated by simultaneously amplifying multiple regions of the nucleic acid using sets of allele-specific primers as described in Wallace et al. (WO89/10414).

Another preferred method for genotyping the SNPs of the present invention is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5,494,810, 5,830,711, and 6,054,564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application PCT/US01/17329 (and Ser. No. 09/584,905) describes OLA (or LDR) followed by PCR, wherein zipcodes are incorporated into OLA probes, and amplified PCR products are determined by electrophoretic or universal zipcode array readout; U.S. applications 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, the primer extension assay involves designing and annealing a primer to a template PCR amplicon upstream (5′) from a target SNP position. A mix of dideoxynucleotide triphosphates (ddNTPs) and/or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions. Mass-tagging also alleviates the need for intensive sample-preparation procedures and decreases the necessary resolving power of the mass spectrometer.

The extended primers can then be purified and analyzed by MALDI-TOF mass spectrometry to determine the identity of the nucleotide present at the target SNP position. In one method of analysis, the products from the primer extension reaction are combined with light absorbing crystals that form a matrix. The matrix is then hit with an energy source such as a laser to ionize and desorb the nucleic acid molecules into the gas-phase. The ionized molecules are then ejected into a flight tube and accelerated down the tube towards a detector. The time between the ionization event, such as a laser pulse, and collision of the molecule with the detector is the time of flight of that molecule. The time of flight is precisely correlated with the mass-to-charge ratio (m/z) of the ionized molecule. Ions with smaller m/z travel down the tube faster than ions with larger m/z and therefore the lighter ions reach the detector before the heavier ions. The time-of-flight is then converted into a corresponding, and highly precise, m/z. In this manner, SNPs can be identified based on the slight differences in mass, and the corresponding time of flight differences, inherent in nucleic acid molecules having different nucleotides at a single base position. For further information regarding the use of primer extension assays in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry”, RAPID COMMUN MASS SPECTROM. 2003; 17 (11):1195-202.

The following references provide further information describing mass spectrometry-based methods for SNP genotyping: Bocker (2003) BIOINFORMATICS 19 Suppl 1:144-153; Storm et al. (2003) METHODS MOL. BIOL. 212:241-62; Jurinke et al. (2002) ADV BIOCHEM ENG BIOTECHNOL. 77:57-74; and Jurinke et al. (2002) METHODS MOL. BIOL. 187:179-92.

SNPs can also be scored by direct DNA sequencing. A variety of automated sequencing procedures can be utilized ((1995) BIOTECHNIQUES 19:448), including sequencing by mass spectrometry (see, e.g., PCT International Publication No. WO94/16101; Cohen et al. (1996) ADV. CHROMATOGR. 36: 127-162; and Griffin et al. (1993) APPL. BIOCHEM. BIOTECHNOL. 38:147-159). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

Other methods that can be used to genotype the SNPs of the present invention include single-strand conformational polymorphism (SSCP), and denaturing gradient gel electrophoresis (DGGE) (Myers et al. (1985) NATURE 313:495). SSCP identifies base differences by alteration in electrophoretic migration of single stranded PCR products, as described in Orita et al., PROC. NAT. ACAD. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel (Erlich, ed., PCR Technology, Principles and Applications for DNA Amplification, W. H. Freeman and Co, New York, 1992, Chapter 7).

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The biological sample for SNP genotyping can be any tissue or fluid that contains nucleic acids. Various embodiments include paraffin imbedded tissue, frozen tissue, surgical fine needle aspirations, and cells of the breast, endometrium, ovaries, uterus, or cervix. Other embodiments include fluid samples such peripheral blood lymphocytes, lymph fluid, ascites, serous fluid, sputum, and stool or urinary specimens such as bladder washing and urine.

### Example 1

The KRAS-variant (rs61764370, GG/TG, LCS6) is a germ-line mutation in a let-7 microRNA-binding site in KRAS, which alters KRAS pathway signaling and let-7 microRNA levels. As demonstrated below, the KRAS-variant can act as a biomarker of altered response in patients with locally advanced head and neck squamous cell carcinoma (HNSCC) treated with radiation and chemotherapy, with or without cetuximab. The impact of the KRAS-variant on outcome in patients who were human papilloma virus (HPV) positive, was also investigated.

As detailed below, of 413 patients tested, 70 (16.9%) had the KRAS-variant. Overall, there was a significant improvement in progression-free survival (PFS) for the first year (HR 0.31, p=0.04) and overall survival (OS) in years 1-2 (HR 0.19, p=0.03) for KRAS-variant patients treated with cetuximab, and no benefit to cetuximab treatment in the non-KRAS-variant group. For patients treated without cetuximab, there was a significant interaction of the KRAS-variant with p16 status (p=0.04). Patients who were KRAS-variant and p16-positive had worse PFS (HR 2.59) and OS (HR 2.48) compared to non-variant patients, yet patients who were KRAS-variant and p16-negative had better PFS and OS (HR 0.62 and 0.61 respectively) than non-variant patients. The addition of cetuximab appeared to improve PFS (HR 0.60) and OS (HR 0.21) for KRAS-variant/p16-positive patients. Immune profiling of HPV positive HNSCC patients indicated that KRAS-variant/p16-positive patients had immune alterations consistent with an immune suppressed baseline, that set them apart from non-KRAS-variant patients.

### Protocol and Patients

NRG Oncology RTOG 0522 was a phase III trial testing the addition of cetuximab to radiation therapy with concurrent cisplatin for patients with advanced HNSCC. 2 Eligible patients had pathologically proven squamous cell carcinoma of the oropharynx, hypopharynx, or larynx, with selected stage III or IV disease (T2 N2-3 MO or T3-4 any N M0), Zubrod performance status 0-1, age≥18 years, and adequate bone marrow, hepatic, and renal function. HPV status was evaluated by p16 expression as previously described.^{1,2}

The UCLA CCRO-022 was a Phase II trial of two cycles of induction paclitaxel and carboplatin chemotherapy followed by radiation and paclitaxel for locally advanced HNSCC associated with human papillomavirus. Eligible patients were patients with stage III or IV, MO squamous cancer of the oropharynx, hypopharynx or larynx that were p16-positive. Zubrod performance status 0-1, age≥18 years, and adequate bone marrow, hepatic, and renal function were also required.

### KRAS-Variant Testing

Genomic DNA from peripheral blood mononuclear cells or whole blood was isolated as previously described for genotyping,³ and 100 ng analyzed in a CLIA-certified laboratory for the KRAS-variant (Mira Dx, New Haven, Conn.). Patients that were homozygous (GG) were grouped with those that were heterozygous (TG) for these analyses.

### Statistical Methods

Local-regional failure (LRF), distant metastasis (DM), progression-free survival (PFS), and overall survival (OS) were as defined in the NRG Oncology RTOG 0522 protocol. LRF and DM rates were estimated by the cumulative incidence method.³² PFS and OS rates were estimated by the Kaplan-Meier method.³³ Hazard ratios were estimated by the Cox model.³⁴ Adverse events were graded by Common Terminology Criteria for Adverse Events (CTCAE) version 3.0. Odds ratios were estimated by logistic regression. Patient characteristics were compared by Fisher's exact test (categorical variables) or Wilcoxon rank-sum test (ordinal or continuous variables). All analyses were performed using SAS version 9.4.

### CCRO HNSCC Patient Immune Phenotyping

Blood samples were analyzed from 26 HNSCC patients that were part of the CCRO study. Up to 40 ml of blood was drawn into heparinized BD Vacutainer® tubes (BD, Franklin Lakes, N.J.) before radiation treatment. Peripheral blood mononuclear cells (PBMCs) were isolated by Ficoll gradient centrifugation within 3 h of blood draw and controlled-rate frozen in aliquots in Fetal Bovine Serum (FBS) containing 10% (v/v) DMSO at −80° C. before transfer to liquid nitrogen until assay.

PBMCs from patients were thawed by dilution in pre-warmed RPMI-1640 medium with 10% (v/v) FBS, treated with DNAse and washed. 4×10⁶ aliquots from each subject were prepared with fixable viability stain 510 (BD Horizon) according to manufacturer's instructions prior to assaying for surface markers as part of a lymphoid panel and a myeloid panel. The lymphoid panel was premixed in brilliant stain buffer (BD Horizon/BD Biosciences) containing FITC anti-human CD4 (clone RPA-T4), PE anti-human CD25 (clone M-A251), PE-CF594 anti-human CXCR3 (clone IC6), PerCP-Cy5.5 anti-human CD3 (clone UCHT1), PE-Cy7 anti-human CD127 (clone HIL-7R-M21), APC anti-human CD45RA (clone HI100), Alexa Flour 700 anti-human CD8 (clone RPA-T8), BV421 anti-human PD-1 (clone EH12.2H7) and BV650 anti-human CCR6 (clone 11A9). For most samples, 1-2×10⁶ cells were stained in 50 μl 2% FBS/PBS staining buffer (BD Pharmingen, San Diego, Calif.) for 20 minutes at room temperature following a 10 minute pre-heat activation at 37° C. in the presence of BV605 anti-human CCR7 (clone 3D12) alone. Cell were washed and re-suspended in 300 μl of PBS and analyzed by flow cytometry within 2 hours. If possible, 2×10⁵ events were accumulated on a LSRFortessa (BD Biosciences, San Jose, Calif.) with UltraComp eBeads compensation (eBioscience, Inc., SanDiego, Calif.).

Analysis was done with FlowJo, LLC (Ashland, Oreg.) using the following gating strategy: 1) FSC-H/FSC-A dot plot to exclude doublets; 2) FVS510-A/FSC-A dot plot to set viability gate; 3) SSC-A/FSC-A dot plot to gate in lymphocyte; 4) CD3/FSC-A dot plot to set gate for CD3⁺ T cells; 5) CD8/CD4 dot plot to select CD3+CD8+ and CD3+CD4⁺ T cells; 5) CD3+CD8+ and CD3+CD4⁺ T cells where individually checked for CCD7/CD45RA expression to dissect naive, effector, central memory and effector memory subsets³⁵ as well as for their PD-1 levels; 7) Regulatory T cells (Tregs) were defined within the CD3+CD4⁺ T cells gate according to CD25^{hi} CD127^{lo} status while the combination of CXC3 and CCR6 guided the distinction between T helper lineages. Quality control required all acquired data to be 250% viability and ≥2,000 CD3⁺CD8⁺ and CD3⁺CD4⁺ T cells.

The myeloid panel comprised FITC anti-human HLA-DR (clone G46-6), PE 5 anti-human CD14 (clone MqP9), PE-CF594 anti-human CD56 (clone BI59), PerCP-Cy5.5 anti-human CD11b (clone ICRF44), PE-Cy7 anti-human CD19 (clone HIB19), APC anti-human CD15 (clone HI98), Alexa Fluor 700 anti-human CD11c (clone B-ly6), APC-H7 anti-human CD20 (clone 2H7), BV421 anti-human CD123 (clone 7G3), BV510 anti-human CD3 (clone UCHT1), and BV650 anti-human CD16 (clone 3G8) premixed in brilliant stain buffer as above. 1-2×10⁶ cells were stained in 50 μl 2% FBS/PBS staining buffer for 30 minutes at room temperature, washed and analyzed as above. The gating strategy was as follows: 1) FSC-H/FSC-A dot plot, gating out doublets; 2) 510-A/FSC-A dot plot to exclude CD3⁺ lymphocytes and dead cells; 3) CD19/FSC-A dot plot to select live CD3CD19 and live CD3CD19⁺ cells with the CD19⁺ subset ultimately giving rise to B cells based on simultaneous CD20 expression; 4) The CD19⁻ subset was used to distinguish between HLA-DR⁺ and DR myeloid lineages; 6) DR⁺ cells led to monocytes subsets according to CD 14/CD16 expression for classical, intermediate and non-classical monocytes, 7) DR⁻ cells on the other hand led us to CD11b⁺CD14^{-/lo}CD15^{hi} granulocytic myeloid-derived suppressor cells (gMDSC); 8) Live CD3⁻ CD19⁻ cells were also used to gate in CD 11b⁺DR^{lo/-}CD14⁺ monocytic myeloid-derived suppressor cells (mMDSC), CD14⁻CD56⁺CD16^{+/-} NK cells as well as dendritic cells of the myeloid (CD11c^{hi}/CD14⁻) and plasmacytoid (CD123^{hi}/CD14⁻) flavor.³⁵

All data were analyzed for statistical significance with a Student's t-test. Statistical significance was at the 5% level.

Results

Clinical Characteristics of Patients with and without the KRAS-Variant

Nine-hundred and forty patients were enrolled into NRG Oncology RTOG 0522, of whom 891 (94.8%) were eligible for protocol analyses, and 413 had biological samples available for KRAS-variant testing (46.4%). Patients not tested for the KRAS-variant had significantly lower age (p=0.02) than patients tested for the KRAS-variant, but the difference in medians was only 2 years (56 vs. 58) (Table 1). PFS and OS were also similar for the patients tested and not tested for the KRAS-variant [PFS hazard ratio 0.92 (95% CI 0.76 to 1.13); OS hazard ratio 0.99 (95% CI 0.78 to 1.25)].

TABLE 1
Pretreatment Characteristics by Whether
or Not KRAS Genotype is Known

| | KRAS genotype known | | | | |
|---|---|---|---|---|---|
| | Yes | | No | | |
| | (n = 413) | | (n = 478) | | p-value |
| Assigned treatment | | | | | 1.0000 [2] |
| No cetuximab | 207 | (50.1%) | 240 | (50.2%) | |
| Cetuximab | 206 | (49.9%) | 238 | (49.8%) | |
| Age (years) | | | | | 0.0159 [3] |
| Mean | 57.5 | | 56.3 | | |
| Std. Dev. | 7.88 | | 8.36 | | |
| Median | 58 | | 56 | | |
| Min-Max | 31-77 | | 34-79 | | |
| Q1-Q3 | 52-63 | | 50-62 | | |
| Gender | | | | | 0.7554 [2] |
| Male | 366 | (88.6%) | 420 | (87.9%) | |
| Female | 47 | (11.4%) | 58 | (12.1%) | |
| Race | | | | | 0.1306 [2] |
| White | 382 | (92.5%) | 428 | (89.5%) | |
| Non-white | 31 | (7.5%) | 50 | (10.5%) | |
| Zubrod performance status | | | | | 0.0895 [2] |
| 0 | 260 | (63.0%) | 327 | (68.4%) | |
| 1 | 153 | (37.0%) | 151 | (31.6%) | |
| Smoking history: pack- | (n = 367) | | (n = 404) | | 0.9281 [3] |
| years [1] | | | | | |
| Mean | 27.4 | | 27.8 | | |
| Std. Dev. | 27.50 | | 28.75 | | |
| Median | 23.5 | | 20.7 | | |
| Min-Max | 0-162 | | 0-150 | | |
| Q1-Q3 | 1-42 | | 0.1-44.9 | | |
| Primary site | | | | | 0.2404 [2] |
| Oropharynx | 298 | (72.2%) | 327 | (68.4%) | |
| Hypopharynx/larynx | 115 | (27.8%) | 151 | (31.6%) | |
| p16 status (oropharynx | (n = 188) | | (n = 133) | | 0.3731 [2] |
| only) | | | | | |
| p16-negative | 54 | (28.7%) | 32 | (24.1%) | |
| p16-positive | 134 | (71.3%) | 101 | (75.9%) | |
| T stage | | | | | 0.3274 [3] |
| T2 | 170 | (41.2%) | 181 | (37.9%) | |
| T3 | 149 | (36.1%) | 180 | (37.7%) | |
| T4 | 94 | (22.8%) | 117 | (24.5%) | |
| N stage | | | | | 0.7947 [3] |
| N0 | 46 | (11.1%) | 53 | (11.1%) | |
| N1 | 36 | (8.7%) | 45 | (9.4%) | |
| N2a | 37 | (9.0%) | 41 | (8.6%) | |
| N2b | 139 | (33.7%) | 154 | (32.2%) | |
| N2c | 137 | (33.2%) | 159 | (33.3%) | |
| N3 | 18 | (4.4%) | 26 | (5.4%) | |

Std. Dev. = standard deviation; Q1 = first quartile; Q3 = third quartile.
[1] A pack-year is defined as the equivalent of smoking one pack of cigarettes a day for 1 year.
[2] Fisher's exact test.
[3] Wilcoxon rank-sum test.

At the time of analysis, median follow-up for surviving patients was 4.8 years (range 0.2 to 6.9). Of the 413 study participants tested for the KRAS-variant, 5 (1.2%) were homozygous (GG) and 65 (15.7%) were heterozygous (TG), resulting in an overall prevalence of 16.9% in this patient population. There was no association of the KRAS-variant and p16 positivity, with the KRAS-variant being found in 17.4% of p16-negative patients and in 16.0% of p16-positive patients, in agreement with prior studies.²³

### Clinical Characteristics in the Cetuximab-Treated Vs Untreated Groups

Within the KRAS-variant cohort, the cetuximab-treated subset had more patients with p16-positive oropharynx tumors than the subset not receiving cetuximab (86.7% vs. 50.0%, p=0.05), and fewer patients of Caucasian ethnicity (87.5% vs. 100%, p=0.04; Table 2). Within the non-variant cohort, the cetuximab-treated subset had significantly lower age than the subset not receiving cetuximab, but the difference in medians was only 2 years (59 vs. 57, p=0.05).

TABLE 2
Pretreatment characteristics by KRAS genotype and assigned treatment

| | Non-variant (TT) | | | | KRAS-variant (GG/TG) | | | |
|---|---|---|---|---|---|---|---|---|
| | No Cetuximab | | Cetuximab | | No Cetuximab | | Cetuximab | |
| | (n = 169) | | (n = 174) | | (n = 38) | | (n = 32) | |
| Age (years) | p = 0.05 | | | | p = 0.22 | | | |
| Mean | 56.7 | | 58.5 | | 57.6 | | 55.6 | |
| Standard deviation | 8.19 | | 7.59 | | 8.29 | | 6.81 | |
| Median | 57 | | 59 | | 58 | | 54.5 | |
| Min-Max | 31-77 | | 41-76 | | 38-75 | | 42-69 | |
| Q1-Q3 | 52-62 | | 53-64 | | 53-63 | | 51-61 | |
| Gender | p = 0.31 | | | | p = 0.44 | | | |
| Male | 146 | (86.4%) | 157 | (90.2%) | 33 | (86.8%) | 30 | (93.8%) |
| Female | 23 | (13.6%) | 17 | (9.8%) | 5 | (13.2%) | 2 | (6.3%) |
| Ethnicity | p = 0.69 | | | | p = 0.04 | | | |
| White | 157 | (92.9%) | 159 | (91.4%) | 38 | (100.0%) | 28 | (87.5%) |
| Non-white | 12 | (7.1%) | 15 | (8.6%) | 0 | (0.0%) | 4 | (12.5%) |
| Zubrod performance status | p = 0.74 | | | | p = 1.00 | | | |
| 0 | 109 | (64.5%) | 109 | (62.6%) | 23 | (60.5%) | 19 | (59.4%) |
| 1 | 60 | (35.5%) | 65 | (37.4%) | 15 | (39.5%) | 13 | (40.6%) |
| Smoking history: pack-years [1] | p = 0.69 | | | | p = 0.14 | | | |
| | (n = 150) | | (n = 154) | | (n = 35) | | (n = 28) | |
| Mean | 26.5 | | 26.7 | | 35.1 | | 26.8 | |
| Standard deviation | 26.08 | | 29.24 | | 26.52 | | 26.10 | |
| Median | 27.25 | | 17.8 | | 34 | | 20.5 | |
| Min-Max | 0-150 | | 0-162 | | 0-90 | | 0-110 | |
| Q1-Q3 | 1.35-40 | | 0-42 | | 4.5-51 | | 3-40.35 | |
| Primary site | p = 0.47 | | | | p = 0.79 | | | |
| Oropharynx | 118 | (69.8%) | 128 | (73.6%) | 29 | (76.3%) | 23 | (71.9%) |
| Hypopharynx/larynx | 51 | (30.2%) | 46 | (26.4%) | 9 | (23.7%) | 9 | (28.1%) |
| p16 status (oropharynx only) | p = 0.29 | | | | p = 0.05 | | | |
| | (n = 77) | | (n = 82) | | (n = 14) | | (n = 15) | |
| p16-negative | 25 | (32.5%) | 20 | (24.4%) | 7 | (50.0%) | 2 | (13.3%) |
| p16-positive | 52 | (67.5%) | 62 | (75.6%) | 7 | (50.0%) | 13 | (86.7%) |
| p16 status (all sites) | p = 0.59 | | | | p = 0.14 | | | |
| | (n = 108) | | (n = 118) | | (n = 22) | | (n = 23) | |
| p16-negative | 50 | (46.3%) | 50 | (42.4%) | 13 | (59.1%) | 8 | (34.8%) |
| p16-positive | 58 | (53.7%) | 68 | (57.6%) | 9 | (40.9%) | 15 | (65.2%) |
| T stage | p = 0.39 | | | | p = 0.24 | | | |
| T2 | 64 | (37.9%) | 74 | (42.5%) | 16 | (42.1%) | 16 | (50.0%) |
| T3 | 64 | (37.9%) | 62 | (35.6%) | 11 | (28.9%) | 12 | (37.5%) |
| T4 | 41 | (24.3%) | 38 | (21.8%) | 11 | (28.9%) | 4 | (12.5%) |
| N stage | p = 0.07 | | | | p = 0.12 | | | |
| N0 | 11 | (6.5%) | 23 | (13.2%) | 5 | (13.2%) | 7 | (21.9%) |
| N1 | 19 | (11.2%) | 14 | (8.0%) | 1 | (2.6%) | 2 | (6.3%) |
| N2a | 11 | (6.5%) | 19 | (10.9%) | 4 | (10.5%) | 3 | (9.4%) |
| N2b | 57 | (33.7%) | 58 | (33.3%) | 12 | (31.6%) | 12 | (37.5%) |
| N2c | 63 | (37.3%) | 54 | (31.0%) | 13 | (34.2%) | 7 | (21.9%) |
| N3 | 8 | (4.7%) | 6 | (3.4%) | 3 | (7.9%) | 1 | (3.1%) |

Q1 = first quartile; Q3 = third quartile.
[1] A pack-year is defined as the equivalent of smoking one pack of cigarettes a day for 1 year.
[P-values for age, pack-years, T stage, and N stage are from Wilcoxon rank-sum test.
P-values for gender, race, Zubrod performance status, primary site, and p16 status are from Fisher's exact test.

KRAS-Variant Status. Cetuximab and Progression Free Survival

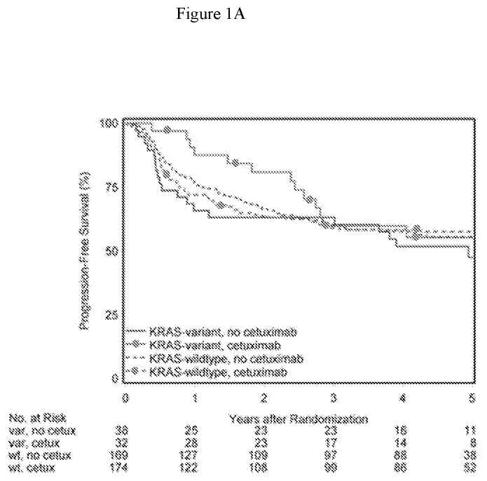

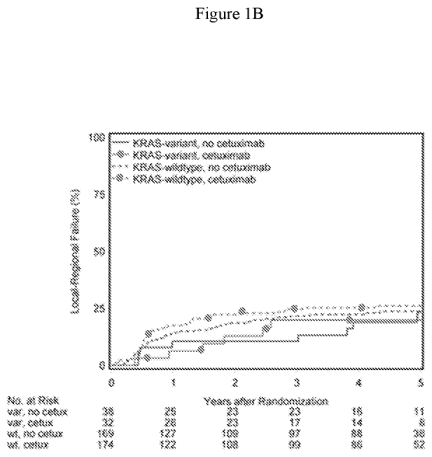

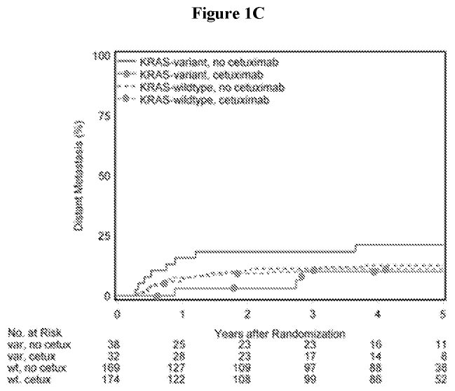

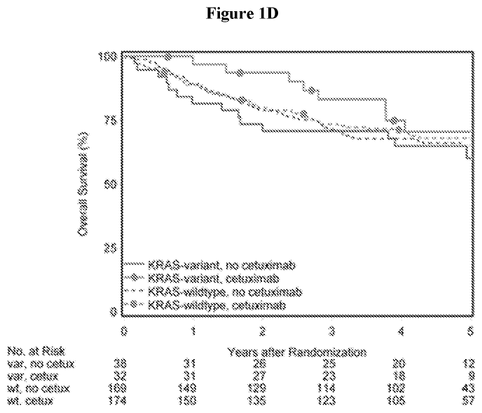

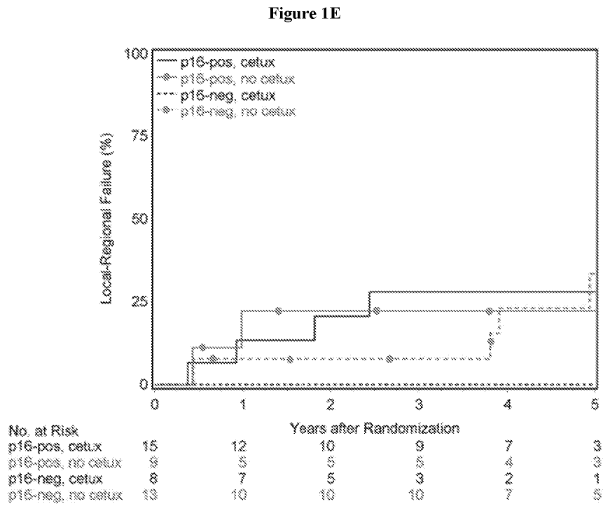

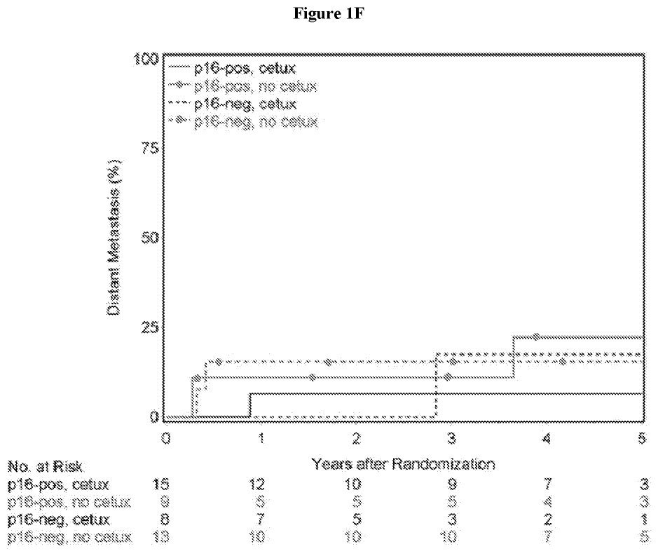

In the KRAS-variant group, a significant, positive effect of cetuximab treatment was found on PFS in the first year (hazard ratio 0.31, 95% CI 0.10 to 0.94, p=0.04), but no significant difference was found after 1 year (hazard ratio 1.76, 95% CI 0.62 to 4.95, p=0.28). For KRAS-variant patients the cetuximab treatment effect significantly varied over time [p=0.02 for interaction between treatment and progression-free survival time (>1 year)]. In the non-variant group there was no impact of cetuximab on PFS with a treatment effect hazard ratio[cetuximab/no cetuximab] of 1.00 (95% CI 0.72 to 1.38, p=0.98). PFS by KRAS-variant status and assigned treatment is shown in FIG. 1A. In multivariate analysis (Table 3) including both KRAS-variant and non-variant patients, the interaction between treatment, time (>1 year), and KRAS group remained significant (p=0.02), indicating that there is an interaction between treatment and time in the KRAS-variant group, but not in the non-variant group. The treatment effect in the first year for the KRAS-variant group was 0.42 (p=0.12) and 1.22 (p=0.64) thereafter (p=0.10 for interaction). In the non-variant group, the treatment effects were 1.20 (p=0.39) and 0.79 (p=0.38) in the first year and thereafter, respectively (p=0.21 for interaction).

TABLE 3
Multivariate analysis of PFS and OS (n = 413)

| | PFS (179 events) | | OS (134 events) | |
|---|---|---|---|---|
| | HR (95% CI) | p-value | HR (95% CI) | p-value |
| Treatment X time X KRAS | — | 0.02 | — | 0.04 |
| Treatment X time, if KRAS-variant | — | 0.10 | — | 0.05 |
| Treatment X time, if non-variant | — | 0.21 | — | 0.80 |
| Treatment X KRAS, if early [1] | — | 0.07 | — | 0.13 |
| Treatment X KRAS, if late | — | 0.36 | — | 0.29 |
| Early treatment effect [1], if KRAS- | 0.42 (0.14-1.26) | 0.12 | 0.27 (0.06-1.21) | 0.09 |
| variant | | | | |
| Late treatment effect, if KRAS-variant | 1.22 (0.53-2.80) | 0.64 | 1.47 (0.53-4.03) | 0.46 |
| Early treatment effect [1], if non-variant | 1.20 (0.80-1.80) | 0.39 | 0.89 (0.56-1.41) | 0.62 |
| Late treatment effect, if non-variant | 0.79 (0.47-1.33) | 0.38 | 0.81 (0.45-1.48) | 0.49 |

PFS, progression-free survival; OS, overall survival; HR, hazard ratio; CI, confidence interval.
[1] First year for PFS, first 2 years for OS.
Hazard ratios estimated from Cox models including treatment (cetuximab vs. no cetuximab), treatment X PFS/OS time interaction, KRAS (variant vs. non-variant), treatment X KRAS interaction, treatment X PFS/OS time X KRAS interaction, age, Zubrod performance status (1 vs. 0), primary site (oropharynx vs. others), T stage (T4 vs. T2-3), and N stage (N2b-3 vs. N0-2a).

Pattern of failure multivariate analysis indicates that DM rather than LRF may be more likely to be contributing to the difference in PFS for KRAS-variant patients: in the KRAS-variant group, the treatment effect for DM is 0.45 (95% CI 0.12 to 1.70) and 0.84 (95% CI 0.29 to 2.42) for LRF. In the non-variant group, the treatment effects for DM and LRF are 0.90 (95% CI 0.48 to 1.70) and 1.24 (95% CI 0.80 to 1.92), respectively. LRF and DM by KRAS-variant status and assigned treatment are shown in FIGS. 1B and 1C.

### KRAS-Variant Status, Cetuximab and Overall Survival

For KRAS-variant patients there was also a significant, positive impact of 8 weeks cetuximab treatment on OS in the first 2 years (hazard ratio 0.19, 95% CI 0.04 to 0.86, p=0.03) but not thereafter (hazard ratio 2.34, 95% CI 0.58 to 9.41, p=0.23). The interaction between treatment effect and survival time (>2 years) was significant (p=0.02), indicating different treatment effects in the first 2 years and thereafter. In the non-variant group, there was no impact of cetuximab treatment on overall survival (treatment effect hazard ratio [cetuximab/no cetuximab] 0.90 (95% CI 0.62 to 1.30, p=0.56). OS by KRAS-variant status and assigned treatment is shown in FIG. 1D. In multivariate analysis (Table 3) including both KRAS-variant and non-variant patients, the interaction between treatment, time (>2 years), and KRAS group is significant (p=0.04) indicating that there is an interaction between treatment and time in the KRAS-variant group but not in the non-variant group. The treatment effect in the first 2 years for the KRAS-variant group is 0.27 (p=0.09) and 1.47 (p=0.46) thereafter (p=0.05 for interaction). In the non-variant group, the treatment effects are 0.89 (p=0.62) in the first 2 years and 0.81 (p=0.49) thereafter (p=0.80 for interaction).

### KRAS-Variant Patients and Toxicity Outcomes

KRAS-variant patients appeared to have similar levels of Grade 3-4 mucositis without or with cetuximab (47.4% vs 50.0%), a difference that was not significant [OR 1.11 (95% CI 0.43 to 2.85), =0.83]. In contrast, the addition of cetuximab increased Grade 3-4 mucositis in non-variant patients from 37.9% to 50.6%, a difference that was significant (OR 1.68 [95% CI 1.09 to 2.58], p=0.02) (Table 4A). However, a test of the interaction between KRAS-variant status, cetuximab treatment and mucositis was not significant (p=0.43). KRAS-variant patients also had similar levels of Grade 3-4 skin reaction inside the portal without or with cetuximab, 18.4% vs 15.6% (OR 0.82 [95% CI 0.23 to 2.89], p=0.76)(Table 4B). In contrast, the addition of cetuximab increased Grade 3-4 skin reaction inside the portal for non-variant patients from 11.2% to 21.8%, a difference that was significant (OR 2.21 [95% CI 1.21 to 4.01], p=0.05), but again a test of interaction was not significant (p=0.16). Both non-variant and KRAS-variant patients developed increased skin reaction outside the portal with cetuximab (non-variant OR 50.15, p<0.001; KRAS-variant OR 8.54, p=0.05) (Table 4C). Although KRAS-variant patients appeared to have higher baseline toxicity in both mucosa and skin inside the portal compared to non-variant patients, these differences were not statistically significant, perhaps because of sample size.

TABLE 4A
Grade 3-4 Treatment-Related [1] Radiation Mucositis
by KRAS-variant and Assigned Treatment

| | | | | Odds Ratio |
|---|---|---|---|---|
| | Assigned | | | (95% Confidence |
| KRAS | Treatment | Patients | Events | Interval) |
| Non- | No cetuximab | 169 | 64 (37.9%) | Reference |
| variant | Cetuximab | 174 | 88 (50.6%) | 1.68 (1.09-2.58) |
| | | | | p = 0.02 |
| Variant | No cetuximab | 38 | 18 (47.4%) | Reference |
| | Cetuximab | 32 | 16 (50.0%) | 1.11 (0.43-2.85) |
| | | | | p = 0.83 |
| Total | | 413 | 186 (45.0%) | interaction p = 0.43 |

TABLE 4B
Grade 3-4 Treatment-Related [1] Skin Reaction Inside Portal
[2] by KRAS-variant and Assigned Treatment

| | | | | | |
|---|---|---|---|---|---|
| Non- | No cetuximab | 169 | 19 | (11.2%) | Reference |
| variant | Cetuximab | 174 | 38 | (21.8%) | 2.21 (1.21 − 4.01) |
| | | | | | p = 0.01 |
| Variant | No cetuximab | 38 | 7 | (18.4%) | Reference |
| | Cetuximab | 32 | 5 | (15.6%) | 0.82 (0.23 − 2.89) |
| | | | | | p = 0.76 |
| Total | | 413 | 69 | (16.7%) | interaction p = 0.16 |

TABLE 4C
Grade 3-4 Treatment-Related [1] Skin Reaction Outside Portal
[3] by KRAS-variant and Assigned Treatment

| | | | | | |
|---|---|---|---|---|---|
| Non- | No cetuximab | 169 | 1 | (0.6%) | Reference |
| variant | Cetuximab | 174 | 40 | (23.0%) | 50.15 (6.81 − 369.54) |
| | | | | | p < 0.001 |
| Variant | No cetuximab | 38 | 1 | (2.6%) | Reference |
| | Cetuximab | 32 | 6 | (18.8%) | 8.54 (0.97 − 75.20) |
| | | | | | p = 0.05 |
| Total | | 413 | 48 | (11.6%) | interaction p = 0.24 |

Odds ratios estimated from logistic regression model with covariates KRAS (variant vs. Non-variant), treatment (cetuximab vs. no cetuximab) and the interaction of KRAS and treatment.
[1] Definitely, probably, or possibly related to protocol treatment.
[2] Dermatitis radiation NOS; Radiation recall syndrome.
[3] Pruritis; Dermatitis exfoliative NOS; Acne NOS; Nail disorder NOS.

### KRAS-Variant, Cetuximab, and p16

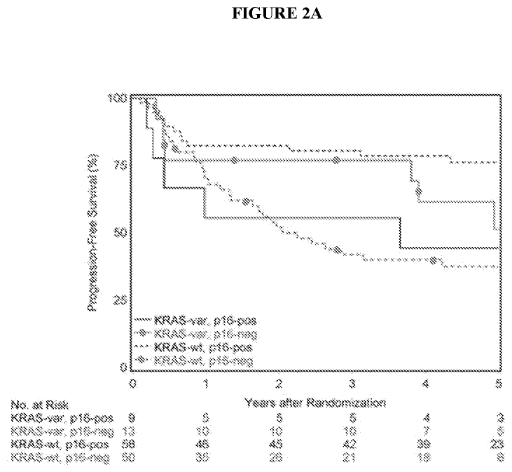

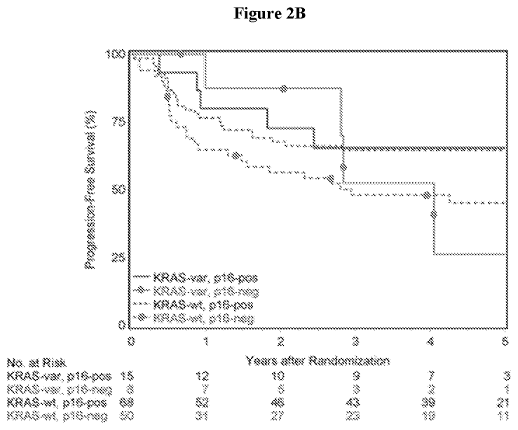

Because of the known prognostic value of p16 and the borderline imbalance in the cetuximab-treated groups, outcome in KRAS-variant patients considering p16 status was evaluated. For PFS, there is some evidence for a three-way interaction between cetuximab treatment, KRAS, and p16 (p=0.20). In patients treated without cetuximab, the two-way interaction between KRAS and p16 was significant (p=0.04). KRAS-variant/p16-positive patients had worse PFS (HR 2.59) compared to non-variant/p16 positive patients. The opposite effect was seen in KRAS-variant/p16-negative patients, who had improved PFS (HR 0.62) compared to non-variant/p16-negative patients (FIG. 2A).

In contrast, 8 weeks of cetuximab treatment appeared to remove the differential effect of p16 on PFS for KRAS-variant patients, (HR 0.89 for p16-positive and 0.77 for p16-negative; p=0.84) (FIG. 2B). The positive impact of cetuximab seemed to be primarily limited to KRAS-variant/p16-positive patients, as the cetuximab treatment effect is 0.60 in KRAS-variant/p16-positive patients, compared to 1.74 in non-variant/p16-positive patients (p=0.14 for interaction between treatment and KRAS). In p16-negative patients, the treatment effects are similar for KRAS-variant and non-variant patients, but may be impacted by time (HR 1.10 and 0.88, p=0.75 for interaction).

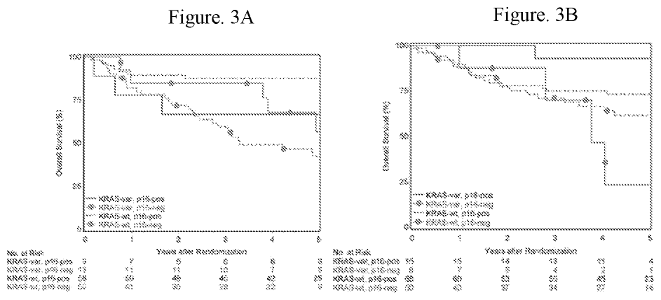

For OS, there is a significant three-way interaction between cetuximab treatment, KRAS-variant status, and p16 (p=0.02). There may be a differential effect of KRAS by p16 in patients treated without cetuximab (p=0.10) and in patients treated with cetuximab (p=0.11). When treated without cetuximab, KRAS-variant/p16-positive patients had worse OS (HR 2.48) compared to non-variant/p16-positive patients. The opposite effect was seen in KRAS-variant/p16-negative patients, who had improved OS (HR 0.61) compared to non-variant/p16-negative patients (FIG. 3A).

For OS, 8 weeks of cetuximab treatment appeared to continue to impact outcome for KRAS-variant patients, with KRAS-variant/p16-positive patients having better OS (HR 0.22) than non-variant/p16-positive patients (FIG. 3B). In p16-positive patients, the treatment effect is 0.21 in KRAS-variant patients compared to 2.36 for non-variant patients (p=0.05 for interaction between treatment and KRAS). The opposite effect was seen in KRAS-variant/p16-negative patients, who appeared to have worse OS with cetuximab addition (HR 1.43), although their OS decreased dramatically over time. In p16-negative patients, the treatment effect is 1.47 in KRAS-variant patients compared to 0.62 in non-variant (p=0.24).

To understand how cetuximab was significantly impacting PFS and OS for KRAS-variant patients, local failure and distant failure for KRAS-variant patients only with or without 8 weeks of cetuximab treatment was evaluated. In KRAS-variant/p16-negative patients, cetuximab appears to decrease local failure, as these patients had no local failures. In KRAS-variant/p16-positive patients there did not appear to be any improvement in local failure with the addition of cetuximab (FIG. 1E). Cetuximab appeared to decrease the rates of distant metastatic failure for KRAS-variant patients who were either p16 positive or negative, which was long lasting for the p16-positive patients, but not the p16-negative patients (FIG. 1F).

KRAS-Variant and the Immunological Landscape

The fact that KRAS-variant/p16 positive patients had significantly improved OS with the addition of cetuximab, but otherwise did significantly worse without cetuximab, possibly through alterations in distant metastatic disease, indicates that KRAS-variant patients may have an inadequate immune response to radiation, that somehow cetuximab helps to overcome. Therefore, the immune system was evaluated in p16-positive HNSCC patients to evaluate for differences in baseline immunity before irradiation in KRAS-variant patients.

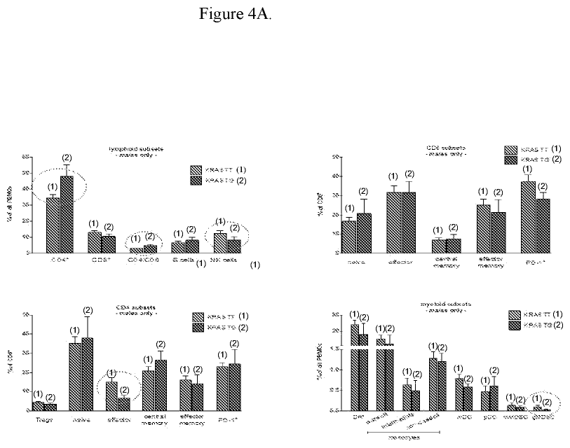

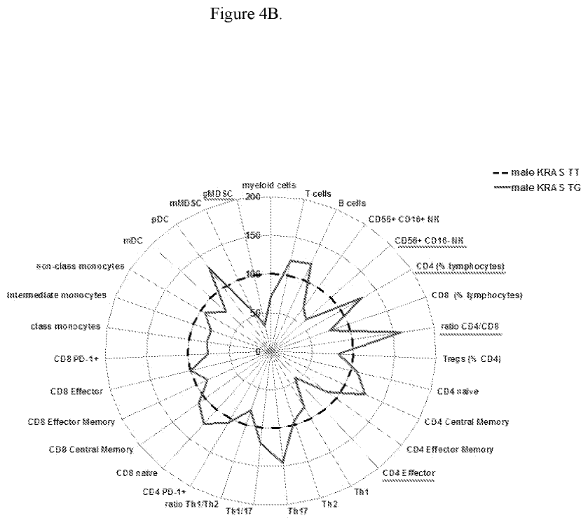

In a cohort of p16 positive advanced HNSCC patients, we performed baseline immune profiling before the beginning of radiation treatment. Owing to the fact that men and women can differ substantially in their immunological make-up, and because the majority of the subjects (21 out of 26; 81%) were men in this study, the analysis was streamlined by including men only. Baseline immunity in men with the KRAS-variant in this cohort (3/21) differed significantly from the rest by having relatively more CD4⁺ PBMCs (p=0.034) as well as a higher CD4/CD8 ratio (p=0.036). In contrast, natural killer cells (NK, p=0.017) and granulocytic myeloid-derived suppressor cells (gMDSCs, p=0.029) were significantly less abundant in KRAS-variant patients (FIG. 4A). Within the CD4 lineage, CD45RA⁺CCR7⁻ effector cells were less frequent (p=0.006) to the advantage of CD45RA⁺CCR7⁺ naive and CD45RA⁻CCR7⁺ central memory subsets, albeit without statistical significance. Hence, it appears that KRAS-variant patients have a broadly shifted immune balance, affecting both lymphoid and myeloid lineages, which appear consistent with a baseline immune deficiency (FIG. 4B).

### Example 2

The following example demonstrates an increased radiosensitivity in KRAS-variant patients. To study the biological and mechanistic basis of the clinical altered radiosensitivity a series of normal and cancer matched, isogenic cell lines, with and without the KRAS-variant, were created by targeted genome editing (Table 5).

TABLE 5

| | KRAS-variant | |
|---|---|---|
| Cell line name | genotype | Tissue Type (origin) |
| MCF10AWT | TT (wild type) | Normal (mammary epithelial) |
| MCF10A MT1 | TG (variant) | Normal (mammary epithelial) |
| MCF10A MT2 | TG (variant) | Normal (mammary epithelial) |
| MCF10A WT + | TT (wild type) | Normal (mammary epithelial) |
| P53 Knockout | | |
| MCF10A MT + | TG (variant) | Normal (mammary epithelial) |
| P53 Knockout | | |
| H1299 WT | TT (wild type) | Cancer (non-small cell lung) |
| H1299 MT | TG (variant) | Cancer (non-small cell lung) |
| HCC1937 WT | TT (wild type) | Cancer (triple negative |
| | | breast) |
| HCC1937 MT | TG (variant) | Cancer (triple negative |
| | | breast) |
| SKOV3 WT | TT (wild type) | Cancer (ovarian) |
| SKOV3 MT | TG (variant) | Cancer (ovarian) |

The positional heterozygous insertion of the KRAS-variant was verified in each isogenic pair by DNA and RNA-sequencing. Allele-specific mRNA expression and analysis of KRAS protein expression by western blot verified bi-allelic expression at the KRAS loci for each isogenic pair. Finally, each isogenic cell line was authenticated by STR analysis at Genetica Laboratories.

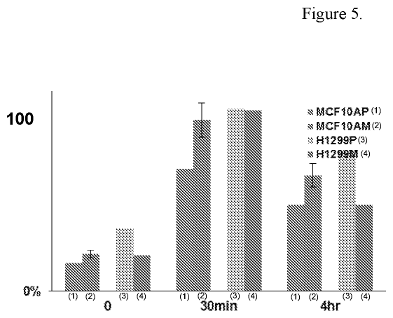

Double strand (DS) break repair is inefficient in KRAS-variant normal tissues versus in KRAS-variant tumor tissues. γH2AX assays were performed to evaluate baseline and residual double strand breaks with and without radiation in the isogenic pairs. MCF 10A and the H1299 cell lines, representing normal and tumor tissues with or without the KRAS-variant were evaluated. Cells were irradiated with 5 Gy, immunofluorescent analysis was performed of FITC conjugated γH2AX by flow cytometry at baseline, at 30 minutes and at 4 hours post radiation (FIG. 5). It was found that in normal tissue (MCF10A, blue bars—labeled with No. 1 and red bars-labeled with No. 2), at all time points there were more double strand breaks in the KRAS-variant cell lines (red versus blue, two separate variant lines averaged, including standard deviation). In contrast, in tumor tissue (H1299, green bars—labeled with No. 3 and purple—labeled with No. 4), it was found that at baseline and at 4 hours there were fewer double strand breaks in the KRAS-variant line (purple versus green). These findings indicate worse DNA maintenance and repair in KRAS-variant normal versus tumor tissues.

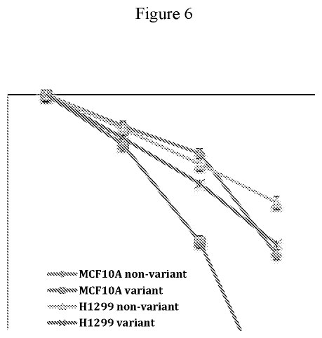

Radiosensitivity of KRAS-variant isogenic normal tissue (MCF10A) and tumor (H1299) cell lines was evaluated using clonogenic cell survival assays in both normal and tumor isogenic cell lines with or without the KRAS-variant. It was observed that the KRAS-variant normal tissue cell line was dramatically radiosensitive as compared to its sister non-variant line. A modest radiosensitivity in the KRAS-variant tumor line was observed as compared to its non-variant sister (FIG. 6).

### Example 3

The following example demonstrates that KRAS-variant patients have a statistically significant reduced likelihood of experiencing a toxic response to an immunotherapy, i.e., an immune related adverse event (irAE). irAEs are discussed in, for example, Abdel-Wahab et al., PLOS ONE, 11(7):e0160221 (2016).

Data was gathered from 88 patients treated with the anti-PD1 antibody therapy Keytruda® (pembrolizumab) or Opdivo® (nivolumab), or with the anti-PDL 1 antibody therapy TECENTRIQ® (atezolizumab). As shown in Table 6, of the 88 patients receiving treatment, 57 patients experienced no toxicity in response to treatment, whereas 31 patients experienced a toxic response. Of the 57 patients experiencing no toxicity, 14 had the KRAS-variant. Of the 31 patients experiencing toxicity 2 had the KRAS-variant. Based on chi-square analysis of these data as shown in Table 6, patients carrying the KRAS-variant were shown to have a statistically significant reduced likelihood of a toxic response to treatment with an immunotherapy as compared to patients not carrying the KRAS-variant given a chi-square value of 4.4268 and a p value of 0.035378 where p<0.05 is significant. Accordingly, patients carrying the KRAS-variant are highly likely to have a non-toxic response to immunotherapy, for example, to checkpoint inhibitor therapies.

TABLE 6
Toxicity: Independent Chi-Square Tests for KRAS

| | | | Total |
|---|---|---|---|
| | KRAS-variant | No KRAS | Patients |
| No toxicity | 14¹ (10.36)² [1.28]³ | 43¹ (46.64)² [0.28]³ | 57 |
| Toxicity | 2¹ (5.64)² [2.35]³ | 29¹ (25.36)² [0.52]³ | 31 |
| Total | 16 | 72 | 88 (Grand |
| Patients | | | Total) |

¹Observed Total # of Patients
²ExpectedTotal # of Patients
³Chi-square statistic

Accordingly, based on this example, a physician can make a determination of whether or not to administer a particular immunotherapy to a patient by determining whether the patient carries a KRAS-variant. The presence of the KRAS-variant would indicate that the patient is likely to have a non-toxic response to the therapy and the patient could be expected to proceed safely with the therapy. This is one important factor for a physician to take into account in determining an appropriate treatment regimen for treating a patient's cancer. In contrast, the absence of a KRAS-variant in a patient would not be determinative of the patient's predicted toxicity to the immunotherapy.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

### INCORPORATION BY REFERENCE

All publications and patent documents cited in this application are incorporated by reference in their entirety for all purposes to the same extent as if the entire contents of each individual publication or patent document was incorporated herein.

### REFERENCES

1. Ang K K, Harris J, Wheeler R, et al: Human papillomavirus and survival of patients with oropharyngeal cancer. N Engl J Med 363:24-35, 2010
2. Ang K K, Zhang Q, Rosenthal D I, et al: Randomized Phase III Trial of Concurrent Accelerated Radiation Plus Cisplatin With or Without Cetuximab for Stage III to IV Head and Neck Carcinoma: RTOG 0522. J Clin Oncol, 2014
3. Bonner J A, Harari P M, Giralt J, et al: Radiotherapy plus cetuximab for squamous-cell carcinoma of the head and neck. N Engl J Med 354:567-78, 2006
4. Vermorken J B, Mesia R, Rivera F, et al: Platinum-based chemotherapy plus cetuximab in head and neck cancer. N Engl J Med 359:1116-27, 2008
5. Fakhry C, Westra W, Li S, et al: Improved survival of patients with human papillomavirus-positive head ane neck squamous cell carcinoma in a prospective clinical trial. J Natl Cancer Inst 100:261-269, 2008
6. Chung C, Zhang Q, Kong C, et al: p16 protein expression and human papillomavirus status as a prognostic biomarker of nonoropharygeal head ane neck squamous cell carcinoma. J Clin Oncol 32:3930-8, 2014
7. Chung Y, Lee M, Homg C, et al: Use of combined molecular biomarkers for prediction of clinical outcomes in locally advanced tonsillar cancers treated with chemoradiotherapy alone. Head Neck 31:9-20, 2009
8. Spanos W, Nowicki P, Lee D, et al: Immune Response During Therapy with Cisplatin or Radiation for Human Papillomavirus-Related Head and Neck Cancer. Arch Otolaryngol Head Neck Surg 135:1137-1146, 2009
9. Pogorzeiski M, Ting S, Gauler T, et al: Impact of human papilloma virus infection on the response of head ane neck cancers to anti-epidermal growth factor receptor antibody therapy. Cell Death Dis 5, 2014
10. Rosenthal D, Harari P, Giralt J, et al: Association of Human Papillomavirus and p16 Status with outcomes in the IMCL-9815 Phase III registration trial for patients with locoregionally advanced oropharyngeal squamous cell carcinoma of the head ane neck treated with radiotherapy with or without cetuximab. J Clin Oncol Dec. 15, 2015
11. Saridaki Z, Weidhaas J, Lenz H-J, et al: A let-7 microRNA-binding site polymophism is KRAS predicts improved outcome in metastatic colorectal cancer (mCRC) patients treated with salvage cetuximab/panitumumab monotherapy. Clin Cancer Res 20:4499-510, 2014
12. Chin L, Ratner E, Leng S, et al: A SNP in a let-7 microRNA complementary site in the KRAS 3 untranslated region increases non-small cell lung cancer risk. Cancer Res 68:8535-40, 2008
13. Paranjape T, Heneghan H, Lindner R, et al: A 3′-untranslated region KRAS variant and triple-negative breast cancer: a case-control and genetic analysis. Lancet Oncol 12:377-86, 2011
14. Ratner E, Lu L, Boeke M, et al: A KRAS-variant in Ovarian Cancer Acts as a Genetic Marker of Cancer Risk. Cancer Research 15:6509-15, 2010
15. Pilarski R, Patel D, Weitzel J, et al: A KRAS-variant is associated with risk of developing double primary breast and ovarian cancer. PLos ONE 7:e37891, 2012
16. Ratner E, Keane F, Lindner R, et al: A KRAS-variant is a Biomarker of Poor Outcome, Platinum Chemotherapy Resistance and a Potential Target for Therapy in Ovarian Cancer. Oncogene Dec. 5, 2011
17. Weidhaas J, Kim E, Herbst R, et al: The KRAS-variant and treatment response in BATTLE-1. J Clin Oncol 32:suppl; abstr 8135, 2014
18. Graziano F, Canestrari E, Loupakis F, et al: Genetic modulation of the Let-7 microRNA binding to KRAS 3′-untranslated region and survival of metastatic colorectal cancer patients treated with salvage cetuximab-irinotecan. Pharmacogenomics J 10:458-64, 2010
19. Zhang W, Winder T, Ning Y, et al: A let-7 microRNA-binding site polymorphism in 3′-untranslated region of KRAS gene predicts response in wild-type KRAS patients with metastatic colorectal cancer treated with cetuximab monotherapy. Ann Oncol 22:104-9, 2011
20. Ganzinelli M, Rulli E, Caiola E, et al: Role of KRAS-LCS6 polymorphism in advanced NSCLC patients treated with erlotinib or docetaxel in second line treatment (TAILOR). Sci Rep 5:16331, 2015
21. Sclafani F, Chau I, Cunningham D, et al: Prognostic role of the LCS6 KRAS variant in locally advanced rectal cancer: results of the EXPERT-C trial. Annals of Oncology 26:9, 2015
22. Christensen B, Moyer B, Avissar M, et al: A let-7 microRNA binding site polymorphism in the KRAS 3′UTR is associatied with reduced survival in oral cancers. Carcinogenesis 30:1003-7, 2009
23. Chung C H, Lee J W, Slebos R J, et al: A 3′UTR KRAS variant is associated with cisplatin resistance in patients with recurrent and/or metastatic head and neck squamous cell carcinoma. Ann Oncol, 2014
24. De Ruyck K, Duprez F, Ferdinande L, et al: A let-7 microRNA polymorphism in the KRAS 3′UTR is prognostic in oropharyngeal cancer. Cancer Epidemiol. 38:591-8, 2014
25. Srivastava R, Lee S, Andrade Filho P, et al: Cetuximab-activated natural killer and dendritic cells collaborate to trigger tumor antigen-specific T-cell immunity in head and neck cancer patients. Clin Cancer Res 19:1858-72, 2013
26. Stephenson R, Lim C, Mattews M, et al: TLR8 stimulation enhances cetuximab-mediated natural killer cell lysis of head and neck cancer cells and dendritic cell cross priming of EGFR-specific CD8+ T cells. Cancer Immunol Immunother 62:1347-1357, 2013
27. Yang X, Zhang X, Mortenson E, et al: Cetuximab-mediated Tumor Regression Depends on Innate and Adaptive Immune Responses. Molecular Therapy 21:91-100, 2012
28. Paranjape T, Heneghan H, Lindner R, et al: A 3′-untranslated region KRAS variant and triple-negative breast cancer: a case-control and genetic analysis. Lancet Oncology 12:377-86, 2011
29. Smakman N, Veenendaal L, van Diest P, et al: Duel effect of KRAS(K12) knockdown on tumorigenesis: increased immune-mediated tumor clearance and abrogation of tumor malignancy. Oncogene 24:8338-42, 2005
30. Sathe A, Ayyar K, Reddy K: MicroRNA let-7 in the spotlight: Role in innate immunity. Inflammation & Cell Signaling e109:1-8, 2014
31. Chin L, Ratner E, Leng S, et al: A SNP in a let-7 microRNA complementary site in the KRAS 3′UTR Increases Non-Small Cell Cancer Risk. Cancer Research 68:8535-40, 2008
32. Kalbfleisch J, Prentice R: The Statistical Analysis of Failure Time Data. New York, Wiley, 1980
33. Kaplan E, Meier P: Nonparametric estimation from incomplete observations. J Amer Stat Assoc 53:457-481, 1958
34. Cox D: Regression models and life tables. J Royal Stat Soc, Series B 34:187-229, 1972
35. Maecker H, McCoy J, Nussenblatt R: Standardizing immunophenotyping for the Human Immunology Project. Nat Rev Immunol 12:471, 2012
36. Sebio A, Pare L, Paez D, et al: The LCS6 polymorphism in the binding site of let-7 microRNA to the KRAS 3′-untranslated region: its role in the efficacy of anti-EGFR-based therapy in metastatic colorectal cancer patients. Pharmacogenet Genomics 23:142-7, 2013
37. John-Aryankalayil M, Palayoor S T, Cema D, et al: Fractionated radiation therapy can induce a molecular profile for therapeutic targeting. Radiat Res 174:446-58, 2010

What is claimed is:

1. A method of treating a cancer subject comprising administering to said subject an immunotherapy in combination with one or more conventional cancer treatments, wherein said administration is dependent on the presence of a KRAS-variant.

2. The method of claim 1, wherein said subject is treated or has been pre-treated with one or more conventional cancer treatments.

3. The method of claim 1, wherein the one or more conventional cancer treatments comprise chemotherapy, radiotherapy, or surgery.

4. The method of claim 1, wherein the cancer is breast cancer, ovarian cancer, non-small cell lung cancer, colorectal cancer or head and neck cancer.

5. The method of claim 1, wherein the method comprises detecting a single nucleotide polymorphism (SNP) at position 4 of the let-7 complementary site 6 of KRAS in a patient sample wherein the presence of said SNP indicates an increased beneficial effect of an immunotherapy for said subject.

6. The method of claim 1 wherein the immunotherapy comprises administration of an immunostimulatory molecule.

7. The method of claim 6, wherein the immunostimulatory molecule is a T-cell activator.

8. The method of claim 6, wherein the immunostimulatory molecule is a dendritic cell activation/maturation factor.

9. The method of claim 1, wherein the immunotherapy comprises administration of a monoclonal antibody, or fragment thereof.

10. The method of claim 9, wherein the monoclonal antibody is specific for an antigen expressed on the surface of a cancer cell.

11. The method of claim 1, wherein the immunotherapy is an anti-EGFR antibody therapy.

12. The method of claim 11, wherein the immunotherapy is cetuximab.

13. The method of claim 5, wherein the single nucleotide polymorphism is a G at position 4 of SEQ ID NO:13.

14.-21. (canceled)

22. A combination drug label wherein said label refers to the use of a drug which as a condition of use must be used in combination with a diagnostic test wherein said diagnostic test is designed to detect a single nucleotide polymorphism (SNP) at position 4 of the let-7 complementary site 6 of KRAS in a patient sample.

23. A reduced-toxicity method of treating cancer, the method comprising administering an immune-modulating cancer therapy to a cancer subject who has been determined to have a single nucleotide polymorphism (SNP) at position 4 of the let-7 complementary site 6 of KRAS.

24. (canceled)

25. The method according to claim 23, wherein the immune-modulating cancer therapy comprises radiation therapy.

26. The method according to claim 23, wherein the immune-modulating cancer therapy comprises a checkpoint inhibitor.

27. The method of claim 26, wherein the checkpoint inhibitor is an antibody to PD-1 or PD-L1.

28. The method of claim 23, wherein the single nucleotide polymorphism is a G at position 4 of SEQ ID NO:13.
